(19) United States
(12) Patent Application Publication
(10) Pub. No.: US 2002/0049157 A1
(43) Pub. Date: Apr. 25, 2002

(54) Use of proteasome inhibitors for treating cancer, inflammation, autoimmune disease, graft rejection and septic shock

(76) Inventors: Jiangping Wu, Brossard (CA); Xin Wang, Montreal (CA)

Correspondence Address: MERCHANT & GOULD PC, P.O. BOX 2903, MINNEAPOLIS, MN, 55402-0903, US

(21) Appl. No.: 09/904,251

(22) Filed: Jul. 12, 2001

Related U.S. Application Data
(63) Continuation-in-part of application No. 09/341,009, filed on Aug. 25, 1999.
(63) A 371 of international application No. PCT/CA98/01010, filed on Oct. 29, 1998.
(60) Provisional application No. 60/218,145, filed on Jul. 14, 2000.

(51) Int. Cl. A61K 38/13; A61K 31/4745

(52) U.S. Cl. 514/9; 514/291

(57) ABSTRACT

The present invention relates to compositions comprising proteasome inhibitors, such as lactacystin, DPBA and their analogs. These compositions are used for the following purposes: (1) to disrupt mitochondrial function (useful aganst cancer, inflammation, adverse immune reaction and hyperthyroidism), (2) to disrupt nitric oxide synthesis (useful against inflammation and septic shock), and (3) to reverse ongoing adverse immune reactions, such as autoimmune diseases and graft rejection. In the later case, the compositions can be administered once the patients' T cells are mostly activated. Proteasome inhibitors can also be combined to immuno-suppressinve drugs like rapamycin, cyclosporin A and FK506. Finally, a method for screening a compound having a proteasome inhibition activity is also disclosed and claimed.

This application is based on U.S. application Ser. No. 60/218,145, filed on Jul. 14, 2000, and is a continuation in part of U.S. application Ser. No. 09/341,009, filed on Jun. 29, 1999, which is a US national application derived from PCT/CA98/01010, filed Oct. 29, 1998.

### FIELD OF THE INVENTION

The present invention relates to the use of proteasome inhibitors for targetting different cellular functions implicated in cancer, inflammation, autoimmune disease, graft rejection and septic shock.

### BACKGROUND OF THE INVENTION

The proteasome is a large protease complex. It is the main nonlysosomal proteolytic system in the cell, and resides in the cytoplasm as well as in the nucleus (Jentsch et al., 1995, Cell 82:881). The proteasome possesses up to five different peptidase activities, in different catalytic domains (Ciechanover, 1994, Cell 79:13; Orlowski et al., 1993, Biochemistry 32:1563), and the best characterized ones are chymotrypsin-like, trypsin-like and peptidylglutamyl-peptide hydrolyzing (PGPH) activities (Orlowski et al., 1981, Biochem & Biophys. Res. Com. 101:814; Wilk et al., 1983, J. Neurochem 40:842).

The proteasome is regarded as a housekeeping enzyme and a “garbage collector” to dispose spent proteins. In fact, the proteasome is responsible for the degradation of 70-90% of cellular proteins (Rock et al., 1994, Cell 78:761). Yet its activity is well controlled and only those destined to be destroyed are timely digested by the proteasome. Through recent studies by the applicants (Wang et al., 1998, J. Immunol. 160:788) and other researchers (Deshaies et al., 1995, EMBO J. 14:303; Yaglom et al., 1995, Mol. & Cell. Biol. 15:731; Seufert et al., 1995, Nature 373:78; Scheffner et al., 1993, Cell 75:495; Pagano et al., 1995, Science 269:682; Palombella et al., 1994, Cell 78:773; Cui et al., 1997, PNAS 94:7515; Treier et al., 1994, Cell 78:787; Lin et al., 1998, Cell 92:819), it becomes increasingly clear that the proteasome plays critical and active roles in regulating many different cellular functions. This is achieved by proteasome's ability to timely, selectively, and irreversibly destroy regulatory protein factors, and by its ability to process precursors of regulatory factors into active ones. For example, the degradation of several important regulators of cell proliferation such as cyclin 2, cyclin 3, cyclin B, p53 and P27^{Kip1} are mediated by the proteasome (Deshaies et al., 1995, supra; Yaglom et al., 1995, supra; Salama et al., 1994, Mol. & Cell. Biol. 14:7953; Seufert et al., 1995, supra; Scheffner et al., 1993, supra; Pagano et al., 1995, supra). The activities of several important regulators involved in cell activation are also controlled by the proteasome. For example, IκBα (Palombella et al., 1994, supra), IκBβ (Cui et al., 1997, supra) and c-Jun protein (Treier et al., 1994, supra) are degraded via the proteasome pathway; the p50 component of a transacting nuclear factor NF-κB matures after cotranslational processing of its precursor peptide by the proteasome (Lin et al., 1998, supra).

According to sedimentation rates, the proteasome could be purified as 26S and 20S complexes. The 20S proteasome is a cylindrical proteolytic core composed of multiple α and β subunits. Each subunit is coded by a different gene in high eukaryotic cells and the total number of subunits varies among different species (Groettrup et al., 1996, Immunol. Today 17:429). In vitro, the purified 20S proteasomes can digest small peptides in an ATP-independent fashion, but they are inactive on intact folded proteins (Peters, 1994, Trends in Biochem. Sci. 19:377). The 20S proteasome can bind at its ends a 19S regulator and forms the 26S proteasome, which degrades ubiquitinated protein in an ATP-dependent fashion (Jentsch et al., 1995, supra). The 20S proteasome can also complex with an 11S activator called PA28 (Groettrup et al., 1996, supra) and form a so-called immunoproteasome (Realini et al., 1994, J. Biol. Chem. 269:20727), which is essential in processing antigenic peptides for presentation by the MHC class I complex. PA28 is a ring-like hexamer or heptamer composed of α and β subunits (PA28α and PA28β), both of which are about 29 KD in size (Realini et al., 1994, supra; Ahn et al., 1995, FEBS Letters 366:37). It is not clear whether the 20S proteasome can associate both the 19S and 11S regulators at the same time.

There are two better characterized mechanisms regulating the protein degradation via the proteasome pathway. The first is that of the substrate selection. This process is controlled by a cascade of enzymes called the ubiquitin-activating enzyme (E1), the ubiquitin-conjugating enzyme (E2) and the ubiquitin ligase (E3) (Jentsch et al., 1995, supra). In addition, the 19S regulator controls the entry of the ubiquitinated protein into the 20S catalytic core. The second mechanism is the activity of the 20S proteasome, which is enhanced by the 11S PA28 (Realini et al., 1994, supra). It is not clear whether and how the 11S PA28 exerts its effect on the 26S proteasome, since it and the 19S regulator do not seem to associate with the 20S at the same time. Moreover, whether the 20S complex exists in parallel to the 26S complex in vivo is still an open question. Nevertheless, it has been shown that overexpression of PA28α could indeed augment significantly antigen processing by the proteasome in vivo (Groettrup et al., 1996, supra). Other controlling mechanism might also exist. For example, a CDK inhibitor p27^{kip1} needs to associate with Jab-1 in order to translocate into the cytoplasm, where it is degraded through the proteasome pathway (Tomoda et al., 1999, Nature 398:160).

Certain peptide aldehydes such as N-acetyl-L-leucinyl-L-leucinal-L-norleucinal (LLnL) and N-carbobenzyoxyl-L-leucinyl-L-leucinyl-L-norvalinal (MG115) are competitive inhibitors of chymotrypsin (Vinitsky et al., 1992, Biochem. 31:9421; Tsubuki et al., 1993, Biochem & Biophys. Res. Com. 196:1195). These agents could effectively block the chymotrypsin-like activity, and to a lesser extent, the trypsin-like and PGPH activities of the proteasome (Rock et al., 1994, supra). They have been employed to study the function of the proteasome in various cellular processes. A caveat of such studies is that these peptide aldehydes are not specific to the proteasome peptidases, and other cellular cysteine proteases such as calpain and cathepsin B (Rock et al., 1994, supra; Sasaki et al., 1990, J. Enzyme Inhib. 3:195) are also potently inhibited. This makes some interpretations less assuring.

Orlowski et al. in U.S. Pat. No. 5,580,854 teach the use of peptidyl aldehydes and their analogues to inhibit proteolysis mediated by the multicatalytic proteinases complex (MPC) or proteasome. The use of such compounds is to inhibit intracellular protein degradation, mitosis and proliferation of dividing cell population. This reference does not teach any apoptotic effect of proteasome inhibitors.

Palombella et al. in WO 95/25533 teach a method for reducing the cellular content and activity of NF-kB, a transcriptional factor playing a central role in immune and inflammatory response, by using proteasome inhibitors, peptidyl aldehydes.

Stein et al. in WO 95/24914 teach a method for reducing the rate of intracellular protein breakdown by inhibiting proteasome activity. The inhibitor MG 101 given as an example is shown to be an inhibitor of 26S proteasome. This inhibitory effect may result in inhibiting destruction of muscle proteins, antigen presentation and degradation of p53.

Omura et al. have reported in 1991 the discovery of lactacystin (LAC) which could induce a neurite outgrowth (Omura et al., 1991, J. Antibiot. 44:113; Ibid., 44:117).

Fenteany et al. have subsequently found that LAC is a proteasome-specific protease inhibitor (Fenteany et al., 1995, Science 268:726). It inhibits the three major peptidase activities (i.e., chymotrypsin-like, trypsin-like, and PGPH activities) of the proteasome, and the inhibition of the first two is irreversible in in vitro assays. LAC does not affect other proteases such as calpain, cathepsin B, chymotrypsin, trypsin, and papain.

Schreiber in WO 96/32105 teaches lactacystin and various analogs to treat conditions that are mediated by the proteolytic function of the proteasome such as rapid elimination and post-translational processing of proteins involved in cellular regulation, intercellular communication and immune response, specifically antigen presentation.

Griscavage et al. (1996, PNAS 93:3308) teach that proteasome activity is essential for the induction of nitric oxide synthase and that the proteasome peptidyl aldehyde inhibitors inhibit the induction of nitric oxide synthase. Nitric oxide production is implicated in initiating and exacerbating symptoms of acute and chronic inflammation (Lundberg et al., 1997, Nature Medecine 3:30-31). Thus the proteasome inhibitors, peptidyl aldehyde, by inhibiting nitric oxide induction have an anti-inflammatory activity. There is no teaching of reproducing the same effect using LAC which is more specific to proteasome than peptidyl aldehydes.

Cui et al. (1997, supra) had shown that T-cell hybridoma can be activated using dishes coated with anti-CD3. Once activated these cells die of apoptosis. It was demonstrated that lactacystin is an inhibitor of activation induced cell death (AICD) and, in these activated hybridoma T-cells, lactacystin must be administered within 2 hours of activation to efficiently block AICD. The same authors state that at higher doses LAC induces apoptosis in the artificial hybridoma T cells.

Grimm et al. (1996, EMBO 15:3835-3844) have shown that proteasome plays a role in thymocyte apoptosis and that peptidyl aldehyde derivatives that inhibit proteasome and LAC block apoptosis in some cases. In addition Grimm et al. (supra) reported that the LAC block of apoptosis was irreversible even when the drug was removed from the cell media. Imajoh-Ohmi et al. (1995, Bioch. Biophys. Res. Com., 217:1070-1077), teach that lactacystin induces apoptosis in human monoblast U937 cells.

The involvement of mitochondria in the apoptotic process has been described by Kroemer et al. (1997, Immunology Today, 18:44). Teachings relating to the mitochondrial control of apoptosis at the induction phase that appear to be essential are provided.

None of these references teach that proteasome inhibitors eliminate activated normal cells. There is no teachings in these references of the involvement of proteasome activity in mitochondrial function. In addition, these references do not describe in mammalian cells what proportion of the protease activity is derived from the proteasome and whether there are efficient and simple methods to screen for additional proteasome inhibitors.

LAC is a specific inhibitor of proteasome, but it is mildly toxic and unstable in aqueous solutions of high pH. LAC and some of its analogues binds directly to the proteasome and inhibits three peptidase activities of the proteasome. However, cellular events downstream of the proteasome are not totally clear. Knowledge of these down stream events related to proteasome activity will allow development of strategies and compounds capable of complementing, synergizing, or substituting the effect of proteasome inhibitors to maximize their effects and/or to minimize their side-effects.

DPBA is also a potent proteasome inhibitor, competitively inhibiting its chymotrypsin-like activity (Palombella et al., 1998, PNAS 95:15671; Adams et al., U.S. Pat. No. 5,780,454). It has a long half-life in aqueous solution (T_{½}=30 days) and Dr. Grisham has shown that, in vivo, DPBA can effectively inhibit Streptococcus cell wall-induced polyarthritis in rats without apparent toxicity (Palombella et al., 1998, supra).

It therefore appears that there is a need to investigate the role of proteasome, namely that of LAC and DPBA and their analogues in the different cellular processes discussed above, and to develop an efficient screening method for searching additional proteasome inhibitors.

The present invention seeks to meet these and other needs.

The present description refers to a number of documents, the contents of which are herein incorporated by reference.

### SUMMARY OF THE INVENTION

Applicants have revealed that PA28 α and β expression is upregulated during T cell activation, and probably as a result, the ex vivo proteasome activity is fourfold higher in the activated T cells than that in the resting T cells (Wang et al., 1996, Eur. J. Immunol. 27:2781). Such an augmented activity likely reflects the increased need to destroy short-lived regulatory proteins and other types of proteins during T cell activation and proliferation. Consequently, it is logical to hypothesize that blocking the proteasome activity will interfere with the activation and proliferation of T cells.

The applicants are the first ones to have documented critical roles of the proteasome in lymphocyte activation and proliferation. They have shown that LAC strongly inhibits mitogen-stimulated T cell proliferation when the compound is added anywhere between the G0 and late G1 phase. This indicates that the proteasome activity is required from the early until the late G1 phase for a successful S phase entry. Mechanistically, the applicants have shown that activation of CDK2 and cyclin E-associated CDK2, which is pivotal for the S phase entry, is proteasome-dependent. Furthermore, it is demonstrated that degradation of a G1 phase CDK inhibitor p27^{kip1} is blocked by LAC. This is a likely mechanism for the inhibition of cyclin E-associated CDK2 by LAC. Additional results have shown that the proteasome inhibition supresses upregulation of p21^{cip1} and CD25 in early G1 phase. These two events are also important for full T cell activation and proliferation (Depper et al., 1983, J. Immunol. 131:690; Labaer et al., 1997, Genes & Development 11:847). Applicants emphasize that the proteasome might also control other cellular events essential for T cell proliferation. In any case, the conclusion of these in vitro results are that proteasome inhibitors can effectively inhibit T cell activation and proliferation. This suggests that such inhibitors can be used as immunosuppressants in the induction phase of organ transplantation.

The invention demonstrates that proteasome is essential for progression of T cells from G₀ to S phase. Taking advantage of LAC's specificity and potency, this compound was used to investigate the role of proteasomes in T lymphocyte activation and proliferation. It is demonstrated that the proteasome is essential for progression of T cells from the Go to S phase. Probably as a result of blockage of cycling, the activated but not resting T cells underwent apoptosis when treated with LAC. It is also shown that the proteasome controls the protein level of p21^{Cip1} and p27^{Kip1} as well as the CDK2 activity in the G₁ phase, and such control mechanism might be essential in the cell cycle progression. LAC can effectively inhibit T cell proliferation even if added at the G₁/S boundary. This knowledge is useful in administering LAC to reverse ongoing graft rejection during the rejection episode.

In addition to inhibiting T cell proliferation, proteasome inhibition causes death of activated but not resting T cells. Applicants are the first to demonstrate this phenomenon. It is shown that LAC can induce apoptosis in cycling Jurkat cells and in mitogen-activated T cells, but not much in resting T cells. Additional mechanistic study by Applicants showed that proteasome inhibition results in reduced degradation of a proapoptic Bcl-2 family member, and the accumulation of Bik contributes the LAC-induced apoptosis. Applicants' results suggest that by inhibiting the proteasome activity, it is possible to clonally delete activated alloantigen-specific T cells in vivo, and achieve long-term graft tolerance.

Thus the present invention relates to inducing apoptosis of activated T cells and T cell leukemia but not resting T cells with LAC or its analogues. Elimination of malignant cells by a proteasome inhibitor-induced apoptosis is useful in cancer therapy. In addition, normal T cells that become activated can be induced to undergo apoptosis with a proteasome inhibitor thus eliminating antigen specific T cells. This is useful in ameliorating autoimmune diseases and graft rejection by generating antigen specific tolerance.

The invention further uses the knowledge of the proteasome involvement in protein degradation and in the steps for the induction of nitric oxide synthase and the effect of LAC or its analogues on the expression of nitric oxide synthase and the production of nitric acid. This is useful in the prevention of septic shock and as an anti-inflammatory.

The present invention also relates to the inhibition of proteasome activity by LAC or its analogues such that the inhibition interferes with cell-cell interaction during lymphocyte activation in mammals and the up-regulation of the adhesion molecule ICAM-1 is repressed. This is useful to control undesirable immune responses during graft rejection, autoimmune diseases and inflammation.

The applicant is the first to show that the electron transport chain in mitochondria is dependent on the intact activity of the proteasome. The addition of proteasome—specific inhibitor such as LAC reduces the electron transport at the complex IV of the respiratory chain. The addition of exogenous cytochrome C reverses this effect. The effect of LAC on mitochondria has potential applications for disorders that relate directly or indirectly to increased activity of mitochondrial function. As well, since proliferating cells have a higher energy requirement, inhibition of mitochondrial respiration could effectively curb the proliferation of cancer cells and activated T cells by depriving the cells of energy, with minimal detriment to normal resting cells.

The applicant is further providing a method for screening proteasome inhibitors by assaying cellular proteinases activity with a tagged peptide substrate. It is understood that this assay protocol can be used in a large through-put screening procedure and that any means of tagging peptide substrates specific to different protease activities of the proteasome and any means for detection known to a person skilled in the art, can be used and incorporated into the large through-put procedure. All the elements comprising a method for screening proteasome inhibitors can be incorporated into a kit.

Applicants are the first to show the dual role of the proteasome in lymphocyte proliferation and apoptosis, which indicates that proteasome inhibitors will be useful immunosuppressants in treating allograft rejection in transplantation. Applicants tested this hypothesis in a mouse heart transplantation model. Since DPBA is more stable than LAC in aqueous solution (Palombella et al., 1998, supra), the Applicants chose the former for this in vivo study.

Therefore, in accordance with the present invention it is provided:

The use of a proteasome inhibitor to induce apoptosis in proliferating cells, wherein said proteasome inhibitor may be lactacystin or an analogue thereof and said proliferating cells are cancerous cells and/or activated T cells, such that activated T cells are antigen induced. The above cells are stopped from progressing from G₀ to G₁/M in a cell cycle as a consequence of proteasome inhibition. As well, CDK2 and the associated Cyclin E activities are substantially inhibited, whereby said cell cycle progression is substantially arrested. Additionally, CDK4 cell activity is not inhibited.

Any one of the use of the above stated provisioned uses of a proteasome inhibitor, wherein said proliferating cells are eliminated and cancer progression is arrested and, activated T cells are eliminated.

The use of a proteasome inhibitor to reverse graft rejection in a patient in need for such a treatment comprising the step of administering to said patient an apoptotic amount of a proteasome inhibitor when said patient T cells are activated wherein said patient is in need of said treatment when an ongoing allograft rejection occurs or at least 24 h after graft transplantation.

The use of a proteasome inhibitor in the making of a medicament to induce apoptosis in proliferating cells. The use of a proteasome inhibitor as defined in the above stated provisions, alone or in combination with another medication, to eliminate or to reduce antigen-specific induced T or B cells, and achieve antigen-specific tolerant status or reduced responsiveness to an antigen in a patient which condition requires such treatment wherein said condition is selected from the group consisting of: autoimmune disease, graft rejection and inflammation.

A method for screening a compound for proteasome inhibition activity, which comprises: obtaining a mammalian cell lysate comprising proteasomes, a partially purified proteasomes preparation or a purified proteasomes preparation; tagging at least one peptide substrate specific to a known proteasome protease activity; combining said proteasomes and said at least one tagged peptide substrate; contacting the so combined proteasomes/tagged peptide substrate with said compound; said at least one tagged peptide substrate fails to release tag if said compound is a proteasome inhibitor, and detecting a decrease or absence of the released tag in the presence of said compound relating to the released tag in the absence of said compound as an indication of proteasome inhibition activity for said compound wherein said at least one tagged peptide substrate is a fluorogenic peptide and wherein said proteasome protease activity is trypsin-like chymotrypsin-like or peptidylglutamyl-peptide hydrolyzing activity.

The use of a proteasome inhibitor to disrupt mitochondrial function, wherein said inhibitor blocks electron transport in said mitochondria and, wherein said inhibitor blocks said electron transport at complex IV in said mitochondria such that mitochondrial function is disrupted, wherein disruption of mitochondrial function is corrected by cytochrome C. The use of the afore-mentioned provisions relating to mitochondrial function to treat a pathological condition wherein high mitochondrial activity occurs, said pathological condition is selected from the group consisting of: cancer, inflammation, undesirable immune responses and hyperthyroidism.

The use of a proteasome inhibitor to disrupt nitric oxide synthesis, wherein the proteasome inhibitor inhibits nitric oxide synthase gene expression.

An apoptotic composition comprising a therapeutically effective amount of a proteasome inhibitor and a pharmaceutically acceptable carrier which may additionally comprise a therapeutically effective amount of an inhibitor to CDK4 activity and/or a therapeutically effective amount of an inhibitor to CDK2 activity and more particularly to Cyclin E activity, a therapeutically effective amount of an inhibitor which prevents p21^{Cip1} upregulation blocks the degradation of p27^{kip1} and a therapeutically effective amount of an inhibitor which prevents CD25 upregulation.

The use of cyclosporin A, rapamycin or FK506 as a proteasome inhibitor.

A composition for use in inhibiting graft rejection comprising a therapeutically effective amount of cyclosporin A, rapamycin or FK506 in combination with a therapeutically effective amount of a proteasome inhibitor and may be in combination with a therapeutically effective amount of an inhibitor of ICAM-1 expression.

A composition for use in inhibiting graft rejection comprising a therapeutically effective amount of an inhibitor which suppresses expression ICAM-1 in combination with a therapeutically effective amount of a proteasome inhibitor.

The use of a proteasome inhibitor to alleviate a disease or a disorder, wherein adhesion molecule ICAM-1 is upregulated and said disease or a disorder is graft rejection, autoimmune disease or inflammation.

The use of a proteasome inhibitor is to alleviate a disease or a disorder wherein at least one of CDK2, p21^{Cip1}, CD25 is upregulated and/or p27^{kip1} degraded, wherein said disease or disorder is graft rejection, autoimmune disease or cancer.

The use of a proteasome inhibitor to alleviate a disease or disorder, wherein nitric oxide synthase is upregulated and said disease or disorder is inflammation or septic shock.

The said proteasome inhibitor may be used alone or in combination with any drugs known in the art for use in treating cancer, inflammation, autoimmune disease, septic shock or inflammation.

The use of all the afore-mentioned provisions wherein said proteasome inhibitor is particularly lactacystin or DPBA or their analogues thereof, is within the scope of this invention. The term “proteasome inhibitor” intends to cover all molecules having the capacity to inhibit the proteasomal enzyme activities. Inhibitors are disclosed in Vinitsky et al., 1992, supra; Tsubuki et al., 1993, supra and Orlowski et al., U.S. Pat. No. 5,580,854. The preferred inhibitors comprises lactacystin and its analogs; examples of such analogs are disclosed in Omura et al., 1991, 44:113; Ibid., 44:117 and in Schreiber, WO 96/32105. The preferred inhibitors also comprise dipeptide boronic acid (DPBA) and its analogs; examples of such analogs are described in U.S. Pat. Nos. 5,462,964, 6,083,903 and in 5,780,454.

### BRIEF DESCRIPTION OF THE DRAWINGS

Having thus generally described the invention, reference will now be made to the accompanying drawings, showing by way of illustration a preferred embodiment thereof, and in which:

FIG. 1 shows that LAC strongly inhibits T and B cell proliferation. Lymphocytes were stimulated with various mitogens as indicated, and LAC at different concentrations was added at the beginning of the cultures. The cells were pulsed with 3H-thymidine between 48 h and 64 h. Samples were in triplicates. All the experiments were performed at least three times and similar results were obtained. Representative results are shown.

A: Peripheral blood T cells stimulated with PHA (2 μg/ml).

B: Peripheral blood T cells stimulated with OKT3 (50 ng/ml).

C: Peripheral blood T cells stimulated with anti-CD28 (50 ng/ml) plus ionomycin (1 μg/ml).

D: Tonsillar B cells stimulated with SAC (1:15,000 dilution) and IL-2 (100 μ/ml).

FIG. 2 shows that inhibition of the proteasome activity results in induction of apoptosis of activated normal cells and leukemic T cells but not resting normal T cells. Tonsillar T cells (A, B, and D) and Jurkat cells (C and E) were treated with LAC (10 μM for T cells and 6 μM for Jurkat cells). LAC was added at the beginning of the culture or 40 h after T cell activation as indicated. The cells were harvested at the time points as shown. They were evaluated for their viability with trypan blue exclusion (A, B, and C), and for their mode of cell death according to DNA fragmentation (D and E).

FIG. 3 shows by electron microscopy that the proteasome inhibitor induced apoptosis in activated T cells and Jurkat cells.

A and B: Morphology of resting T cells treated with LAC. Tonsillar T cells were culture in the absence (A) or presence (B) of LAC (10 mM) for 24 h, and the cells were examined by EM.

C and D: Morphology of activated T cells treated with LAC. Tonsillar T cells were first activated with PHA (2 μg/ml) for 40 h. The cells were then cultured in the absence (C) or presence (D) of LAC (10 μM) for additional 24h, and were examined with EM.

E and F: Morphology of Jurkat cell treated with LAC. Jurkat cells were cultured in the absence (E) or presence (F) of LAC (6 μM) for 24 h and were evaluated with EM. Arrows indicate condensed nuclei.

FIG. 4 shows that the effect of LAC is rapid and reversible in cell culture.

A. The rapid effect of LAC Peripheral blood T cells were pretreated with 10 μM LAC in culture medium or in culture medium alone for 3 h or 16 h. The cells were then washed and recultured in the presence of 2 μg/ml PHA for 64 h. The cells were pulsed with ³H-thymidine for 16 h before they were harvested at 64 h. Samples were in triplicates.

B. The inhibitory effect of LAC on the proteasome activity was reversible in the cells Jurkat cells were pretreated with LAC (6 μM) in culture medium for 3 h. The cells were washed and recultured at 0.5×10⁶ cells/ml for 0 h, 5 h or 21 h. The cells were then harvested, washed and sonicated. The lysate protein (20 μg/sample) was assayed for its proteinase activity under a condition at which 90% of the activity was attributed to the proteasome. The samples were in duplicates. The result is expressed as relative fluorescence intensity at 440 nm.

C. The activity of LAC in culture supernatants is short-lived LAC (6 μM) was added to Jurkat cell culture (0.5×10⁶ cells/ml). The supernatants were harvested at 4 h, 6 h, 16 h and 24 h. These conditioned media were used to culture fresh Jurkat cells for 3 h. The cells were then harvested and assayed for the proteasome activity as described in FIG. 4B. Samples were in duplicates.

All the experiments were performed at least three times, and similar results were obtained. Representative data are shown.

FIG. 5 shows that LAC inhibits CD25 upregulation during T cell activation.

Peripheral blood T cells were stimulated with PHA (2 μg/ml) for 48 h in the presence or absence of LAC (10 μM, added at the beginning of the culture). CD25 expression on T cells was evaluated by anti-CD25-PE/anti-CD3-FITC two-color flow cytometry. Similar results were obtained in two independent experiments, and a representative one is shown. The data are presented as two color histograms in forms of contours, as well as in an overlay histogram.

FIG. 6 shows the role of the proteasome in cell cycle progress.

A. LAC does not inhibit the progress from the G₂/M phase to the G₁ phase in synchronized Jurkat cells Jurkat cells were synchronized at the G₂/M phase by 16 h nocodazole treatment. For the last 3 h of the treatment, LAC (6 μM) was added to the cultures destined to be treated by LAC later. The cells were then released by washing out nocodazole, and recultured in complete medium with or without 6 μM LAC. The cells were sampled at 0 h, 4 h and 8 h after the G₂/M release, stained with propidium iodide, and analyzed with flow cytometry.

B. LAC slows the cell cycle progress from the G₁/S boundary to the G₂/M phase in synchronized Jurkat cells Jurkat cells were synchronized at the G₁/S by isoleucine starvation followed by a hydroxyurea treatment. The synchronized cells were released by washing out hydroxyurea and were cultured in complete medium in the absence or presence of LAC (6 μM). The cells were sampled at 0 h, 3 h, 6 h, 9 h, 12 h, 15 h and 24 h after the release, and were stained with propidium iodide and analyzed with flow cytometry.

C and D. LAC blocks the S phase entry of the mitogen-stimulated peripheral blood T cells Peripheral blood T cells were stimulated with PHA (2 μ/ml) in the absence or presence of LAC (10 μM, added at 0 h, 16 h, 24 h, or 40 h, as indicated in the bottom of the panels). For the flow cytometry analysis of the cell cycle progress, the cells were harvested at 0 h, 1 6 h, 40 h and 64 h as indicated on the top of the panels (FIG. 6C). For ³H-thymidine uptake, the triplicated cell samples were pulsed at 48 h and harvested at 64 h (FIG. 6D).

The experiments were performed three times, and similar results were obtained. Representative data are shown.

FIG. 7 shows the results of the kinase assays for the effect of LAC on CDK activity.

Tonsillar T cells were activated with PHA (2 μg/ml) for a period as indicated in each graph. LAC (10 μM) was added once at 0 h. The cells were harvested at 16 h, 24 h, or 40 h as indicated. An equal amount of lysate protein (40 μ/sample) was precipitated with rabbit anti-CDK4, anti-CDK2 or anti-Cyclin E antisera (2.5 μg Ab/sample). The immune complexes were assayed for their kinase activities using histone H1 as a substrate. (A) CDK4 kinase activity. (B) CDK2 kinase activity. (C) Cyclin E-associated CDK activity. The membrane in (C) was subsequently hybridized with anti-Cyclin E (1 μg/ml) followed by ¹²⁵I-protein A for the evaluation of the protein level of Cyclin E.

All the experiments were performed three times, and similar results were obtained. Representative data are shown.

FIG. 8 shows the results of immunoblotting analysis of the effect of LAC on the protein levels of Cyclin E and Cyclin A.

Tonsillar T cells were stimulated with PHA (2 μg/ml) for 40 h in the presence of hydroxyurea (1 mM), and these cells were blocked at the G₁/S boundary (G₁ block). The synchronization was released by washing out hydroxyurea, and the cells were recultured in medium containing 2 μg/ml PHA in the absence or presence of LAC (10 nM, added once at the time of the release). The cells were harvested at 6 h and 22 h post the G₁/S block. The cell lysates (40 μg/sample) were resolved in 10% SDS-PAGE, and transferred to PVDF membranes. The membranes were hybridized with rabbit-anti-Cyclin E or anticyclin A antisera followed by ¹²⁵I-protein A. The Cyclin E level (FIG. 8A) and cyclin A level (FIG. 8B) of representative experiments are shown. Similar results were obtained in a total of three independent experiments.

FIG. 9 shows the results of immunoblotting analysis of the effect of LAC on the levels of CDK inhibitors P27^{Kip1} and p21^{Cip1}.

Tonsillar T cells were stimulated with PHA (2 μg/ml) for 16 h, 40 or 64 h in the absence or presence of LAC (10 μM). For the 16 h and 40 h culture, LAC was added once at 0 h. For the 64 h culture, LAC was added once at 40 h. The cell lysates were resolved in 10% SDS-PAGE, and blotted onto PVDF membranes. The membranes were hybridized with rabbit anti-p27^{Kip1} antisera (FIG. 9A) or with anti-p21^{Cip1} antisera (FIG. 9B) followed by ¹²⁵-protein A. The experiments were performed three times, and similar results were obtained. Representative data is shown.

FIG. 10 shows human peripheral blood mononuclear cells that were cultured in medium (A), 2 μg/ml PHA (B), or PHA plus 10 μM lactacystin for 24 h. Lactacystin could effectively block the aggregate formation.

FIG. 11 shows mouse lymph node cells that were cultured in medium (A), 2 μg/ml Con A (B), or Con A plus 10 μM lactacystin for 24 h. Lactacystin could effectively block the aggregate formation.

FIG. 12 shows mouse lymph node cells from TCR transgenic mice named 2C that were cultured in medium (A), 2 μg/ml Con A (B), or Con A plus 10 μM lactacystin. After 24 h and 48 h, the cells were examined for ICAM-1 expression by flow cytometry, using FITC-anti-ICAM-1/1B2-PE. Monoclonal Ab 1B2 recognize a clonotypic determinant on the TCR of the transgenic T cells which are largely CD8 positive (>75%). Lactacystin could effectively block the upregulation of ICAM-1 on those CD8 positive T cells.

FIG. 13 shows mouse peritoneal exudate macrophages that were stimulated with 2 μg/ml LPS in the presence of lactacystin at different concentrations. Nitric oxide production by the macrophages was measured according to the nitrate concentrations in the supernatants.

FIG. 14 shows mouse peritoneal exudate macrophages that were stimulated with 2 μg/ml LPS in the presence or absence of lactacystin (10 μM). Nitric oxide synthase expression was measured with Northern blot analysis.

FIG. 15 shows that Lactacystin blocks electron transport downstream of Complex I. Respiration of Jurkat cells (JC) or rat kidney mitochondria (RKM) was measured by O₂ consumption using an oxygen electrode. The function of Complex I of digitonin (Dig)-permeated Jurkat cells was blocked by rotenone (Rot), and the respiration was resumed by adding succinate (Suc), which provides electrons to Complex II directly and thus bypasses Complex I. The maximal respiration was achieved by adding CCCP (carbonyl cyanide m-chlorophenylhydrazone), which uncouples the oxidation and phosphorylation. The respiration could be blocked by antimycin A (Ant), which inhibits Complex II. Curves 1 and 6 represent positive controls of rat kidney mitochondria. Curves 2 and 5 represent normals untreated Jurkat cells. Curves 3 and 4 represent Jurkat cells treated with lactacystin (6 μM) for 2 h and 4 h, respectively.

FIG. 16 shows that Lactacystin blocks electron transport at Complex IV. Complex III in the respiration chain was blocked at Complex III antimycin (Ant), and the electron flow was resumed by addiind ascorbate (Asc) and TMPD (tetramethyl-p-phenyl-enediamine). The maximal respiration was triggered by CCCP, and was totally inhibited by potassium cyanide (KCN).

FIG. 17 shows that Cytochrome completely corrects the defect at Complex IV caused by LAC. The assay system is identical to that described in FIG. 16. Jurkat cells were treated with LAC for 4 h (curve 3). The decoupling reagent used in this experiment to achieve maximal respiration is FCCP (carbonylcyanide-p-trifluoromethoxyphenylhydrazone).

FIG. 18 shows that RAPA, FK506, and CsA inhibit PA28 expression at the mRNA level. Tonsillar T cells (A) and B cells (B) were cultured in the presence of various reagents as indicated (PHA, 2 μg/ml, RAPA, 10 nM; FK506, 10 nM, CsA, 1 μM; SAC, 1:10,000 dilution; Il-2, 25 U/ml. After 6 h, 20 h or 40 h, the cells were harvested and total RNA was analyzed by Northern blotting for PA28β expression. The PA28β message in T cells was also examined by Northern blotting using a similar condition as for PA28β (C). The experiments were repeated more than three times, and representative ones are shown.

FIG. 19 shows that RAPA inhibits PA28β and PA28α protein in the activated T cells. (A) An analysis of PA28β protein by immunoblotting is shown. Tonsillar T cells were cultured with 2 μg/ml PHA or PHA plus 50 nM RAPA for 24 h. The cells were harvested and lysed. Forty micrograms of cleared lysate protein per sample was analyzed by immunoblotting using rabbit anti-PA28β antiserum. (B) An analysis PA28α and PA28β protein by confocal immunofluorescence microscopy. Tonsillar T cells were cultured with 2 μg/ml PHA or PHA plus 50 nM RAPA for 24 h. The cells were stained with antisera specific for PA28α and PA28β. Thirteen cells were analyzed for PA28α protein and twelve cells for PA28β protein in a blind fashion. The mean+SD of relative fluorescence intensity per whole cell is presented. Unpaired Student's t-test was employed for statistics. The difference between PHA-activated sample and PHA plus RAPA-treated samples was highly significant (p=3.20×10⁹ for PA28α and p=5.99×10⁻⁵ for PA28β).

FIG. 20 shows that effect of RAPA on proteasome activity in human PBMC. Human PBMC were cultured in the absence or presence of 2 μg/ml PHA or 10 nM RAPA for 16 h-70 h as indicated. The cells were then harvested, and the chymotrypsin-like activity of whole cells lysates was assayed in the absence or presence of 20 μM proteasome inhibitor LAC. The data are presented as arbitrary units of fluorescence intensity per 20 μg lysate protein. The experiments were repeated three times and a representative one is shown. Samples are in duplicate and the mean±SD is shown. (A) Total chymotrypsin-like activity in the lysate of PBMC. (B) Lactacystin-inhibitable chymotrypsin-like activity in the lysate of 70 h PBMC. Nine micrograms of 20S proteasome were used as positive controls for the inhibitory effect of LAC at 10 μM and 20 μM. LAC was always added to the lysates during the proteinase assay 15 min before the addition of the substrate. The solid bars represent the activity in the presence of LAC. The net proteasome activities are calculated as the total activity minus the remaining activity after the LAC addition.

FIG. 21 shows the elimination of an alloantigen-specific response by a proteasome inhibitor lactacystin. The C57BL/6 spleen cells (H-2^{b}) were stimulated with mitomycin c-treated BALB/c spleen cells (H-2^{d}). On day 2 when most of the H-2^{d}-specific cells were activated, the mixed lymphocyte culture (MLR) was treated with lactacystin (LAC, 8 μM) for 3 h. After wash, the cells were put back in culture for additional 8 days, and then stimulated with either fresh BALB/c or C3H (H-2^{k}) spleen cells. In MLR treated by LAC, the C57BL/6 cells failed to respond to the BALB/c cells, but respond well to third party C3H (H-2^{k}) cells. The difference is more pronounced in day three of the culture.

FIG. 22 shows that the LAC-induced DNA fragmentation is inhibited by a broad spectrum caspase inhibitor zVAD.fmk. Jurkat cells were treated with LAC (6 μM) in the absence or presence of different concentrations of zVAD.fms (0.4 μM to 33.3 μM) for 6 h. The cells were harvested and their DNA was analyzed by a DNA fragmentation assay according to DNA laddering.

FIG. 23 shows that preventing the degradation of a pro-apoptotic Bcl-2 family member Bik is a mechanism for the proteasome inhibitor-induced apoptosis. Jurkat cells were treated with lactacystin (6 μM) for 5 h (lanes 2 and 4 of panel A), 4 h (lane 2 of panel B) or 7 h (lane 3 of panel B), lane 1 in panels A and B is untreated control samples. The cells were separated into mitochondrial (mito in panel A and mitochondria in panel B) and cytosolic (cytosol in panel A) fractions, and the lysate of these two fractions analyzed by immunoblotting using goat anti-Bik, and rabbit anti-Bax, Bak and Bad Ab (all from Santa Cruz Biotech, Santa Cruz, Calif.) followed by enhanced chemiluminescence (ECL, kit from Amersham).

FIG. 24 shows that overexpression of an anti-apoptotic Bcl-2 family member Bcl-xL in a B cell line could protect the cells from apoptosis caused by proteasome inhibition. A human B cell line Namalwa was stably transfected with an anti-apoptotic Bcl-2 family member Bcl-xL, and its sensitivity to the proteasome inhibitor-induced apoptosis tested by the quantitative filter elution assay (Schmitt et al., 1998, Exp. Cell Res. 240:107), which detects DNA fragmentation during apoptosis. The wild type Namalwa and transfected Namalwa cells overexpressing Bcl-xL were pulsed with ¹⁴C-thymidine for 24 h, and then treated with different concentrations of lactacystin (0.75 μM, 1.5 μM, 3 μM, 6 μM and 10 μM). The cells were harvested at different time intervals (24-96 h), and DNA fragmentation measured.

FIG. 25 shows that the wild type Namalwa cells have increased Bik level after treatment with lactacystin and that the Bcl-xL transfected Namalwa cells have overexpressed Bcl-xL. Jurkat cells, wild type Namalwa cells and Bcl-xL transfected Namalwa cells were treated with medium (lanes 1), staurosporine (0.3 μM, lanes 2) and lactacystin (6 μM, lanes 3) for 6 H. The proteins from the mitochondrial fraction of these cells were analyzed by immunoblotting and the amount of Bik, Bcl-xL, Bax, and Bak evaluated. The same membranes were used sequentially and probed with different antibodies against these factors. A nonspecific band recognized by a monoclonal antibody against cytochrome oxygenase (COX) was used as control for even sample loading in the lanes.

FIG. 26 shows the chemical structure of the proteasome inhibitors dipeptide boronic acid (DPBA; Pyz-Phe-boroLeu; Pyz, 2, 5-pyrazinecarboxylic acid) and lactacystin.

FIG. 27 shows the inhibition of the 20S protesome activity by the proteasome inhibitor DPBA. The 20S proteasome was purified from rat liver as described in the applicant's previous publication (1996, supra). A fluorogenic peptide sLLVY-MCA was used as a chymotrypsin substrate. DPBA of different concentrations was added into the reaction mix, and incubated at 37° C. for 30 min. The relative fluorescent intensity, which reflects the chymotrypsin-like enzymatic activity of the 20S proteasome, was measured with a fluorometer using excitation/emission wavelengths of 380 nm/440 nm.

FIG. 28 shows the suppression of anti-CD3-stimulated T cell proliferation by the proteasome inhibitor DPBA. BALB/c mouse spleen cells were stimulated with anti-CD3 (clone 2C11, 50 ng/ml), and DPBA of different concentrations was present in the culture. The cells were pulsed with ³H-thymidine at 48 h and harvested at 64 h after the culture.

FIG. 29 shows that the proteasome inhibitor DPBA prolongs mouse heart allograft survival. BALB/c mice (H-2^{d}) were used as heart donors and C57BL/6 mice (H-2^{b}) as recipients. Heterotopic heart transplantation was performed on day 0, and a proteasome inhibitor DPBA was administrated from day 1 to day 16 i.p. daily. Group 2 was given 0.65 mg/kg/day; group 3 was given 1.0 mg/kg/day for 4 days, and the dose was then reduced to 0.5 mg/kg/day for 12 days. The graft survival days, mean survival time (MST) and the p value (unpaired Student's test) compared with the control group was presented.

FIG. 30 shows that the proteasome inhibitor DPBA is effective in treating ongoing heart allograft rejection in mice. The experiment was carried out as described in FIG. 29, except that DPBA was only administrated between day 3 and 6 for 4 days, when the rejection is ongoing.

FIG. 31 shows that the proteasome inhibitor DPBA effectively prevents mouse islet allograft rejection. C57BL/6 mice were treated with 250 mg/kg streptozocin and used as islet graft recipients when their blood glucose reached 20 nM. Islets from BALB/c mice were isolated after collagenase digestion followed by Ficoll gradient separation. The islets were cultured overnight, and transplanted into the peritoneal cavity of the diabetic C57BL/6 recipients (500-600 islets/recipient). Twenty-four hours after the transplantation, the recipients were given DPBA i.p. at 1 mg/kg/day for 16 days and then at 0.5 mg/kg twice a week until day 60 post operation. The blood glucose of the mice was measured daily and the means+SDs are shown. The isograft controls are diabetic C57BL/6 mice transplanted with C57BL/6 islets (500-600 islets/recipient), and were not treated with DPBA. The allograft controls were diabetic C57BL/6 mice transplanted with BALB/c islets (500-600 islets/recipient) without DPBA treatement. The mice were sacrificed on day 60, or when their blood glucose reached 20 nM (the allograft control group).

Other objects, advantages and features of the present invention will become more apparent upon reading of the following non-restrictive description of preferred embodiments with reference to the accompanying drawings which are exemplary and should not be interpreted as limiting the scope of the present invention.

### DESCRIPTION OF THE PREFERRED EMBODIMENTS

The present invention relates to proteasome activities in cellular processes and any inhibitors of proteasome activities.

Proteasome Activity is Obligatory for Activation and Proliferation of T and B Cells

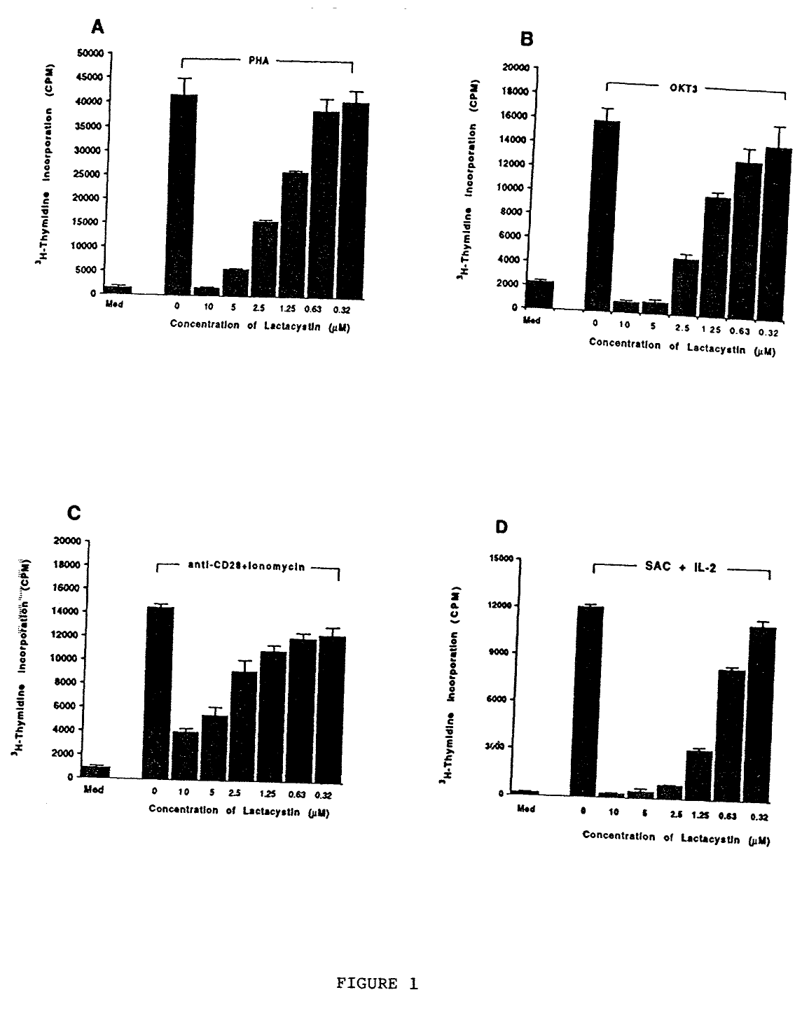

The role of proteasome in T cell activation and proliferation was first examined in PBMC, using the proteasome-specific inhibitor LAC. The PBMC were activated with various stimulants. LAC was added to the cells in the beginning of the culture (0 h) along with the stimulants. ³H-thymidine uptake between 48 h and 64 h of 64 h cultures was used as a parameter for cell proliferation. As shown in FIG. 1, LAC strongly and dose-dependently inhibited the T cell proliferation induced by a T cell mitogen PHA (FIG. 1A), by crosslinking TCR with anti-CD3 E (FIG. 1B), or by Ca⁺⁺ ionophore plus cross-linking of the T cell co-stimulating molecule CD28 (FIG. 1C). The T cell-independent B cell proliferation induced with SAC plus IL-2 in tonsillar B cells was also potently inhibited by LAC (FIG. 1D). In all the four systems employed, LAC at 5 μM could exert near-to-maximal inhibition. The results suggest that LAC's effect is not lymphocyte type (T or B cells)-specific nor stimulant-specific. Rather, it likely affects certain downstream events governing a more general process(es) in lymphocyte activation and proliferation.

LAC Causes Apoptosis in Activated but not Resting T Cells

In one embodiment of the present invention a compound is provided that induces activated and leukemic T cells to undergo apoptosis.

Since LAC has been reported to induce apoptosis in U937 cells (Chen et al., 1996, J. Immunol. 157:4297), it is crucial to examine whether the LAC-induced inhibition of cell proliferation is due cell death, be it apoptosis or necrosis.

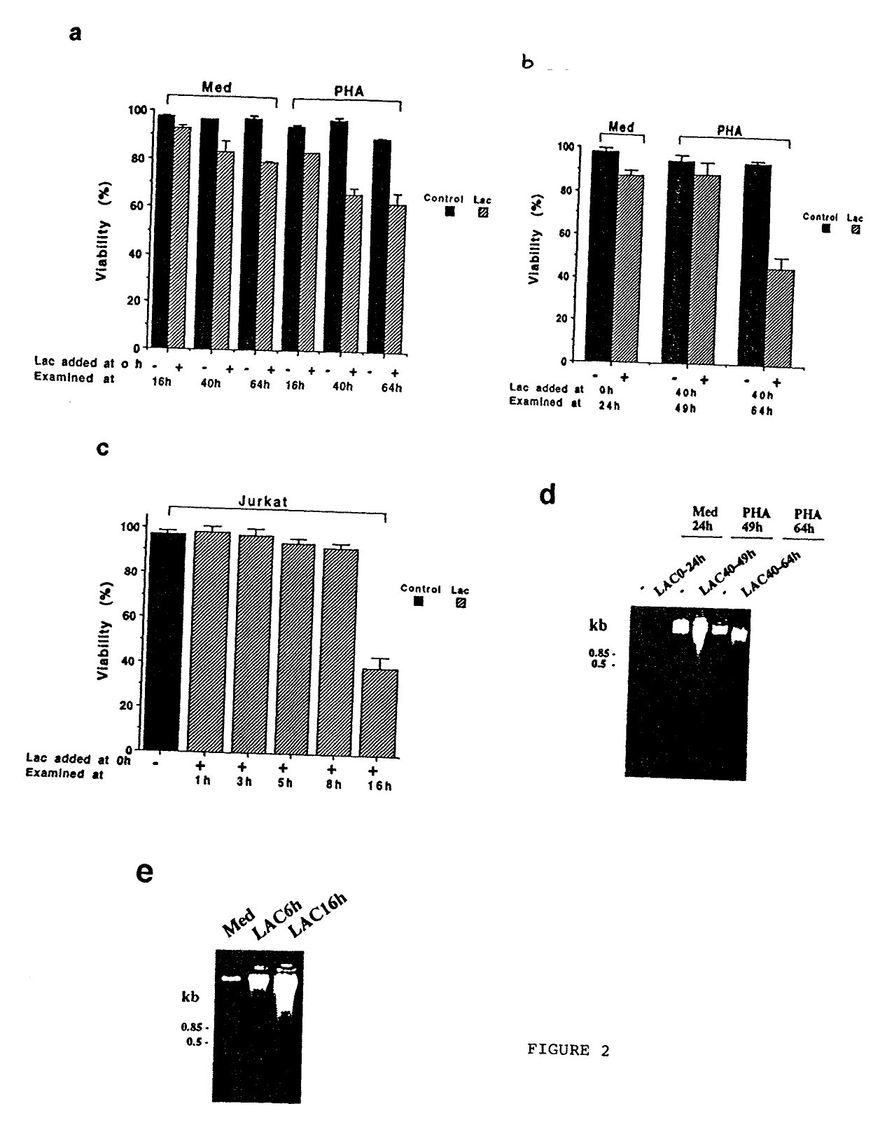

The viability of T cells and Jurkat cells after LAC-treatment was first evaluated with trypan blue exclusion. Resting T cells (T cells in medium) or PHA-stimulated T cells were cultured with 10 μM LAC (LAC added at the beginning of the culture). As shown in FIG. 2A, after 16 h culture, the viability of the cells only had minor decreases (<12%) in LAC-treated cells compared with those without LAC (97% vs 92% for cells in medium, and 94% vs 83% for cells with PHA). After a prolonged culture for 64 h, the decreases were more prominent although were still less than 27% (97% vs 79% for cells in medium, and 90% vs 63% for cells with PHA).

There was a tendency that the activated T cells were more susceptible to LAC than the resting T cells. This became more evident when LAC was added to T cells 40 h after the PHA activation (FIG. 2B). The viability of the activated T cells dropped from 94% to 46% after additional 24 h culture, although 9 h culture did not change the viability significantly according to trypan blue exclusion. On the other hand, the viability of the resting T cells in medium had only a small decrease (from 98% of the control to 87% of the LAC-treated) after 24 h of LAC presence.

Why did LAC added at 0 h along with PHA cause less cell death compared with LAC added at 40 h post PHA stimulation (FIG. 2A vs 2B)? It will be demonstrated that LAC is rapidly degraded in the cell culture. After 24 h in culture medium, LAC lost its activity, and at 40 h when the T cells were fully activated and become more susceptible, there was no biologically active LAC in the culture. This could explain the observed difference in terms of viability between the 0 h and 40 h addition of LAC to the PHA-activated T cells.

The effect of LAC on Jurkat cells was quite similar to that on the activated T cells. Less than 8 h exposure to 6 μM LAC did not induce apparent Jurkat cell death, while about 60% of the Jurkat cells were trypan blue positive after 24 h culture with LAC (FIG. 2C).

We next employed DNA laddering to study the mode of cell death caused by LAC, and the result of this experiment also reflected the degree of cell death after different treatments. As shown in FIG. 2D, resting T cells treated with 10 μM LAC for 24 h had no apparent DNA breakdown (lanes 1 and 2). This correlated to the good cell viability as shown in FIG. 2B. On the other hand, clear DNA ladders could be observed from activated T cells (40 h post PHA-stimulation) treated with LAC for additional 9 h (lanes 3 and 4). After 24 h of LAC treatment, the ladders became less discrete, and this probably reflected further DNA breakdown. For Jurkat cells, DNA fragmentation could be detected as early as 6 h after the LAC treatment, and after 16 h, the fragmentation became more prominent (FIG. 2E).

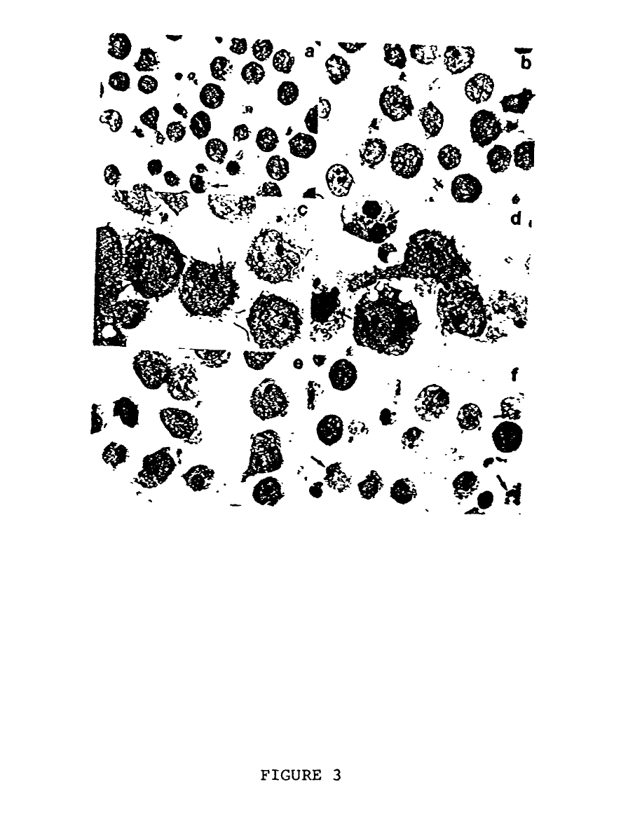

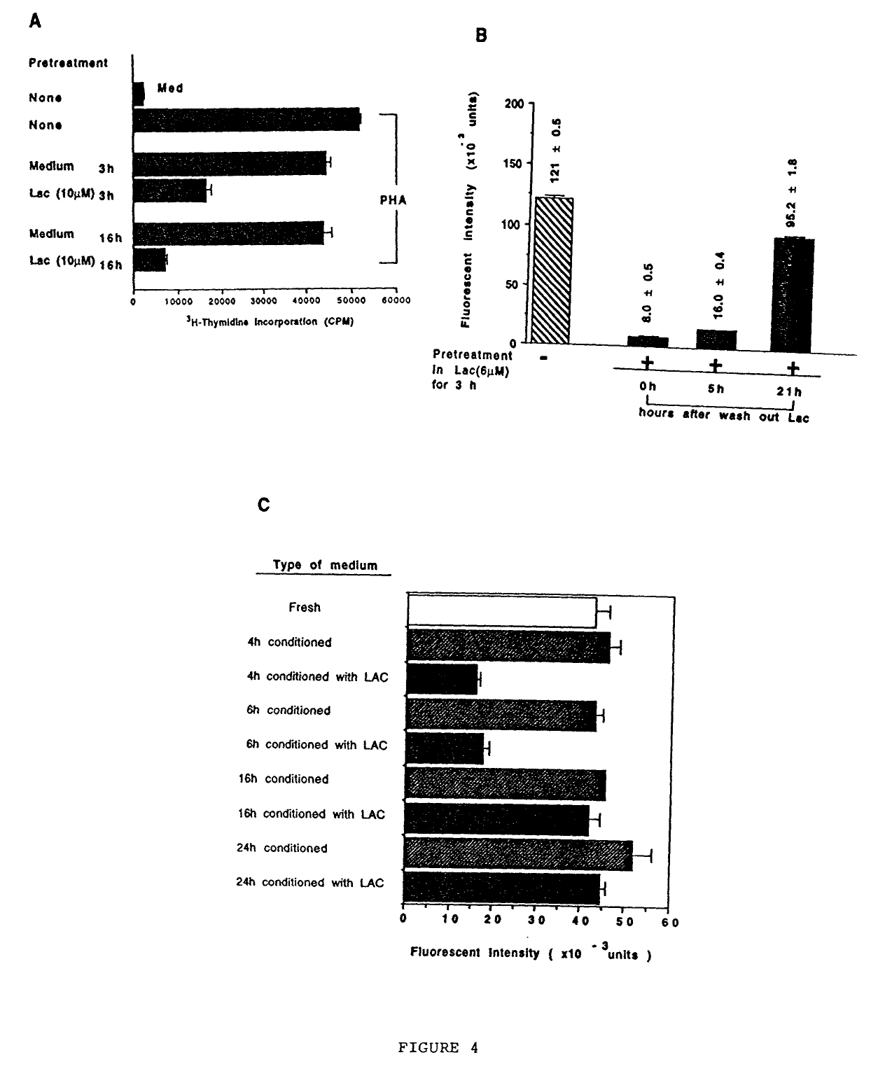

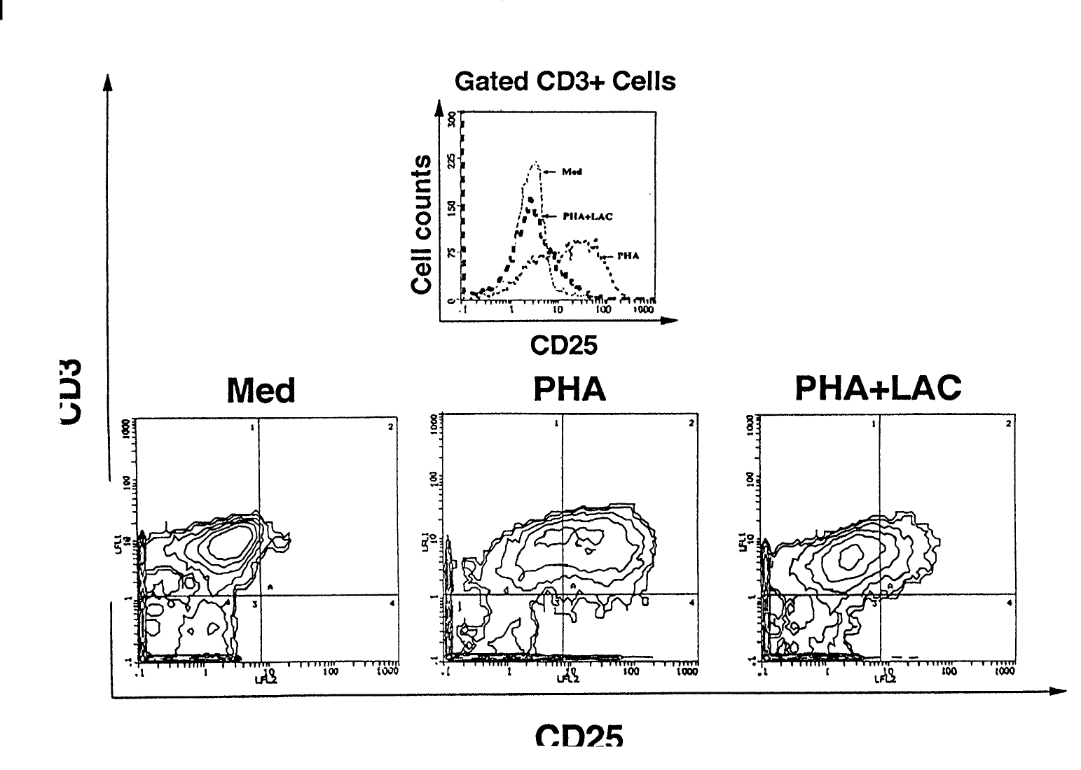

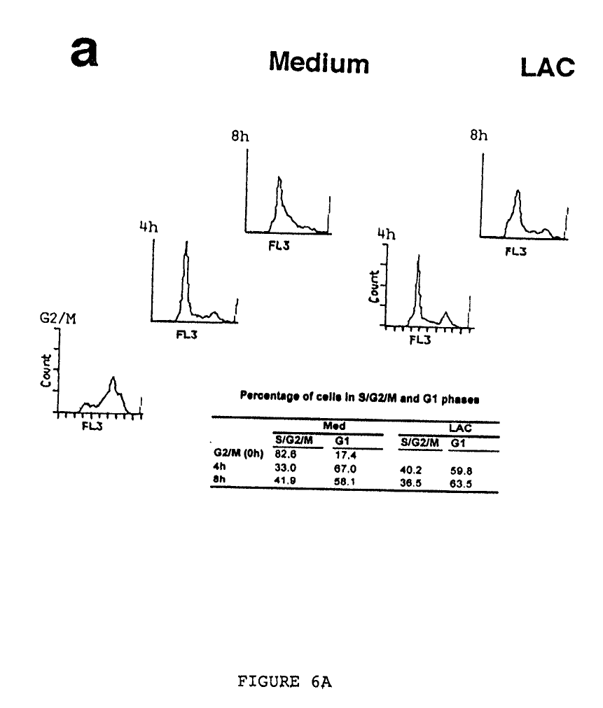

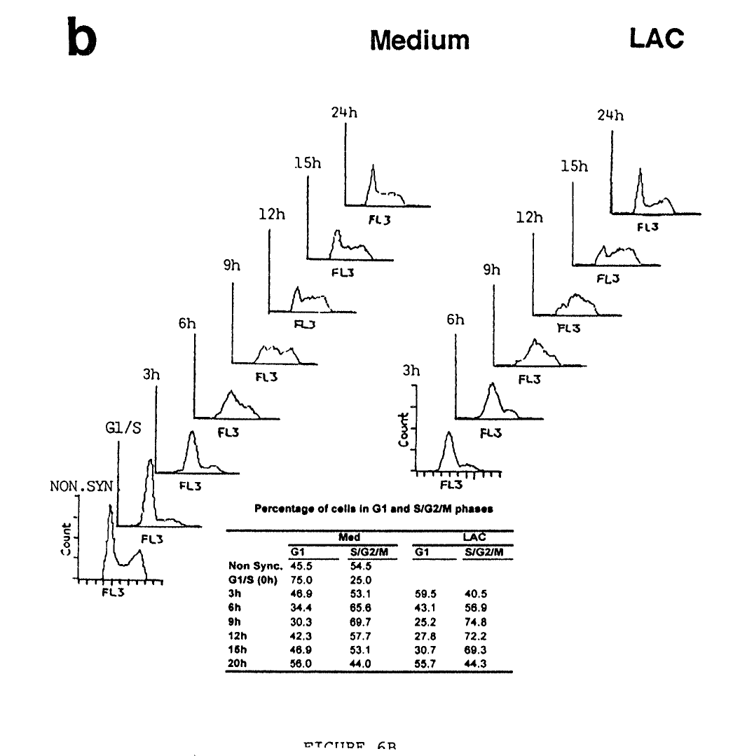

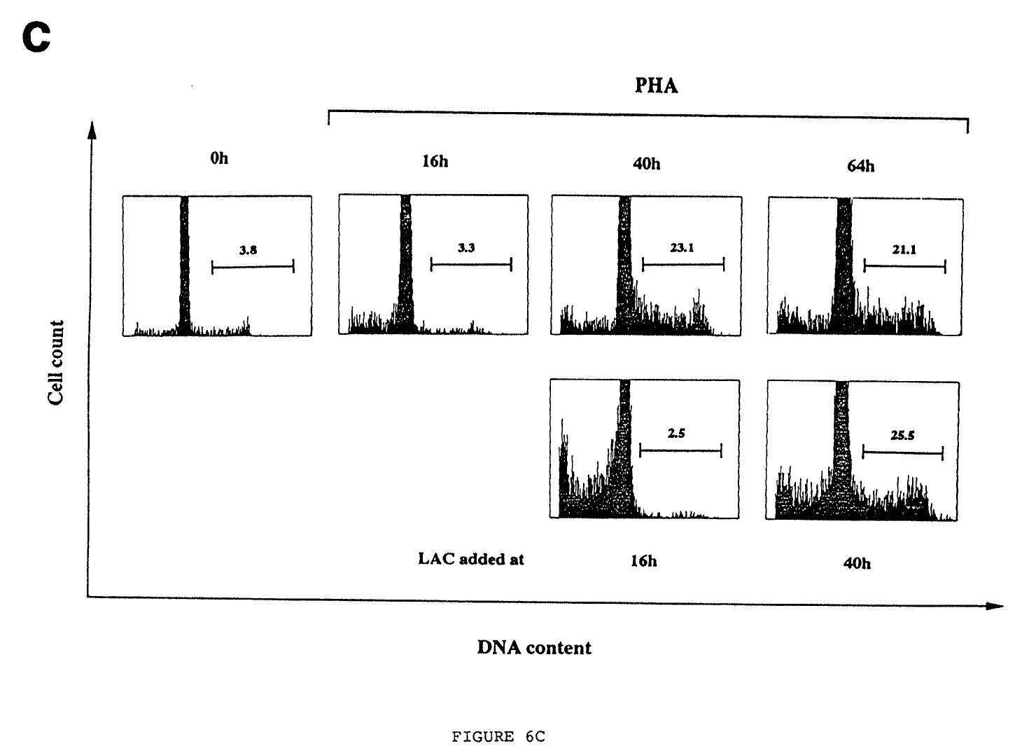

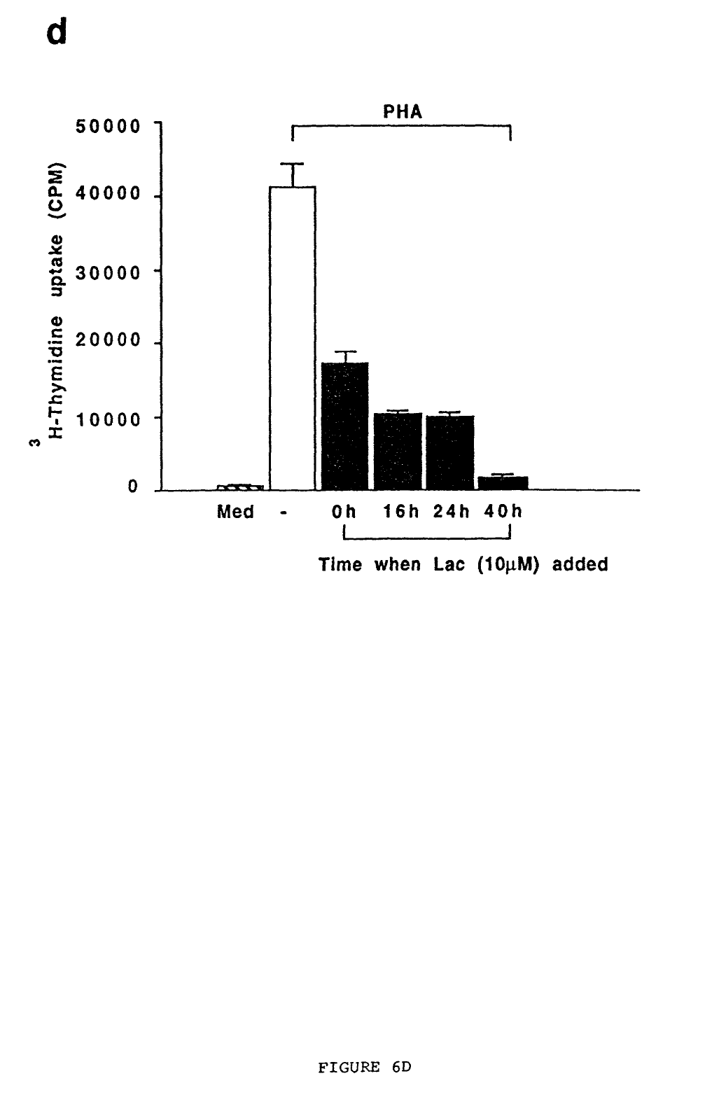

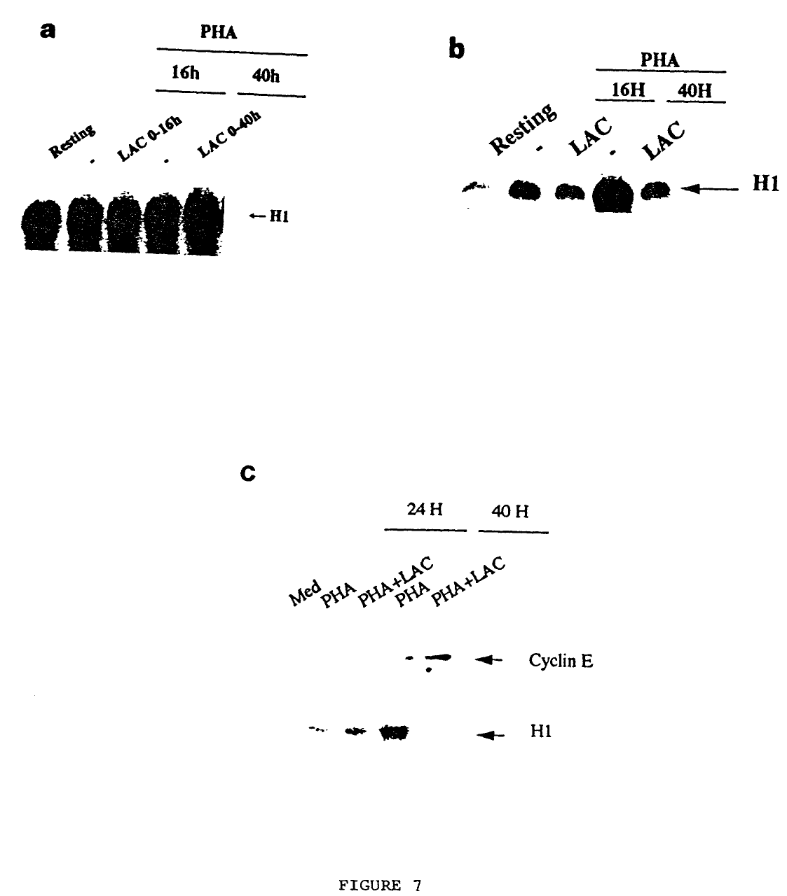

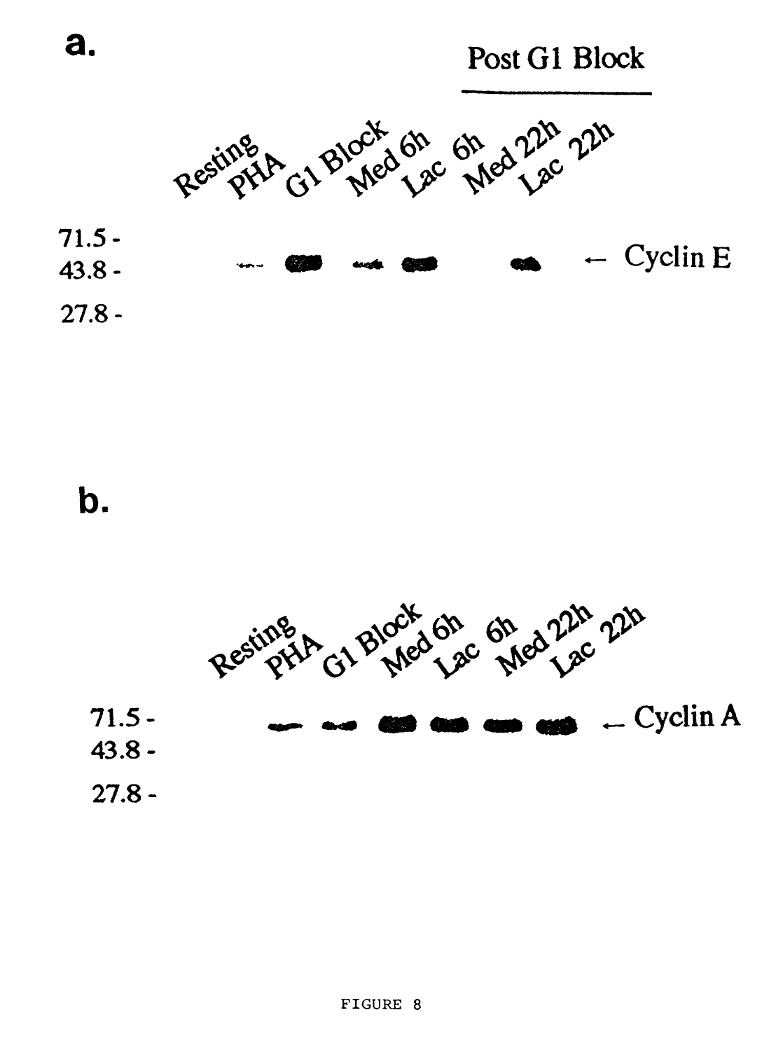

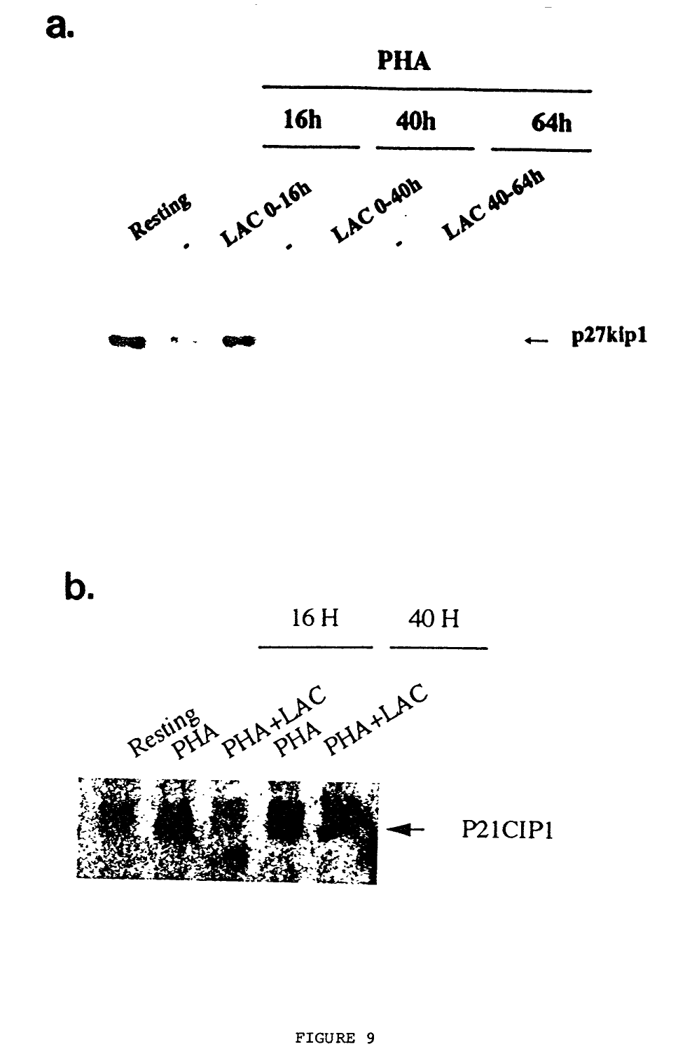

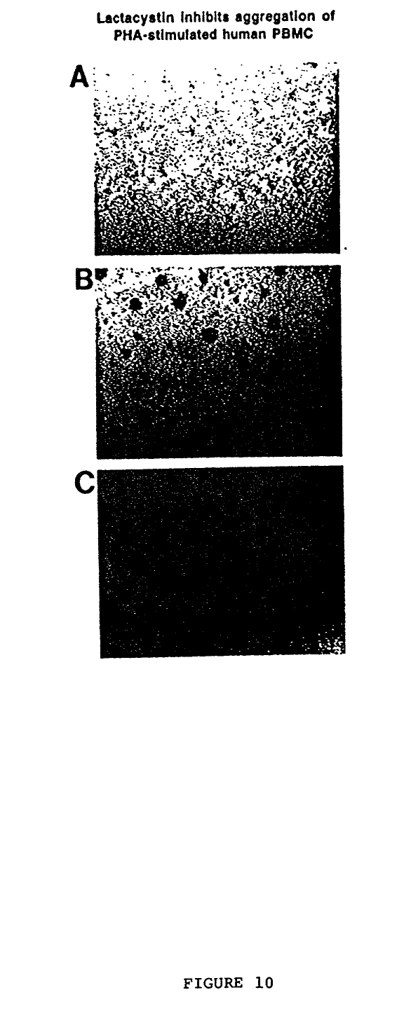

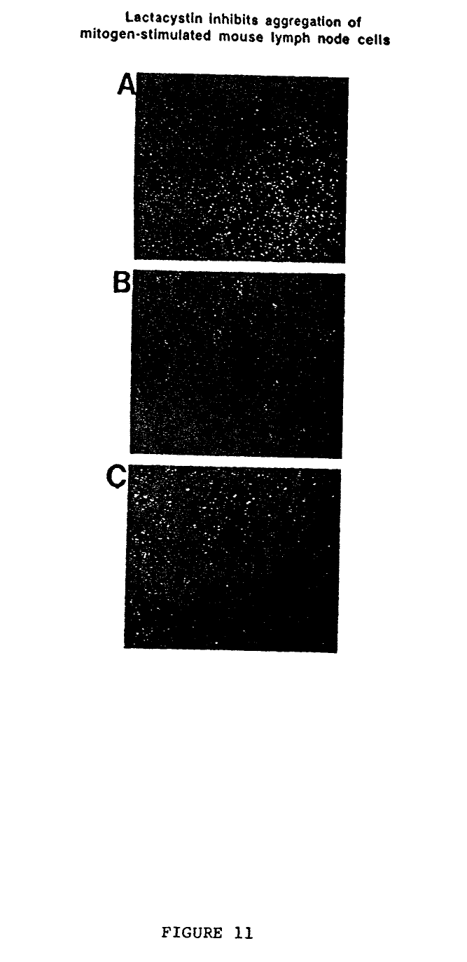

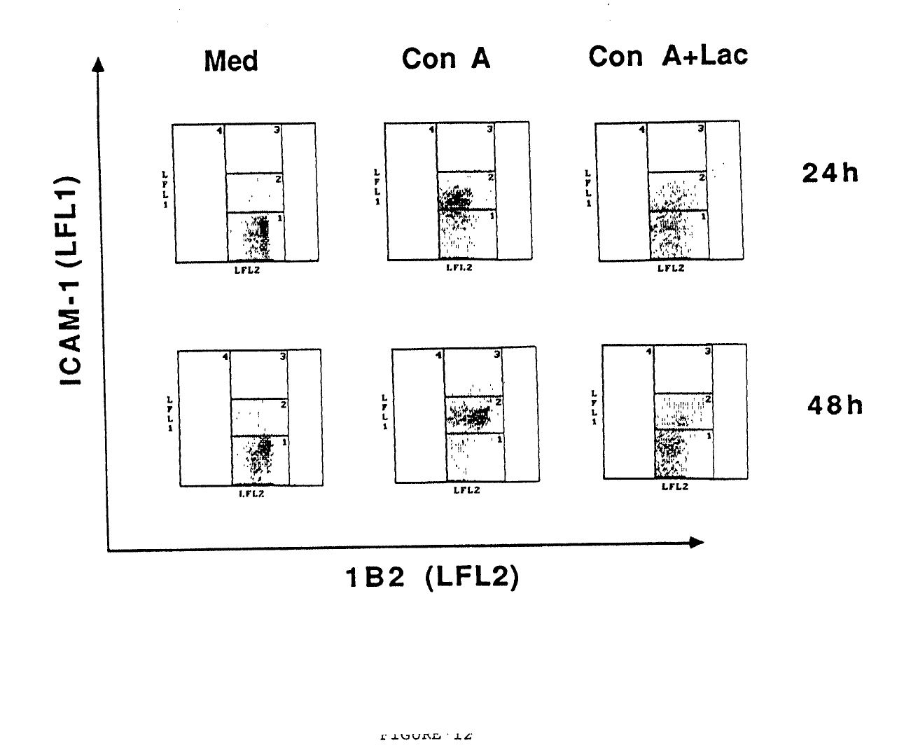

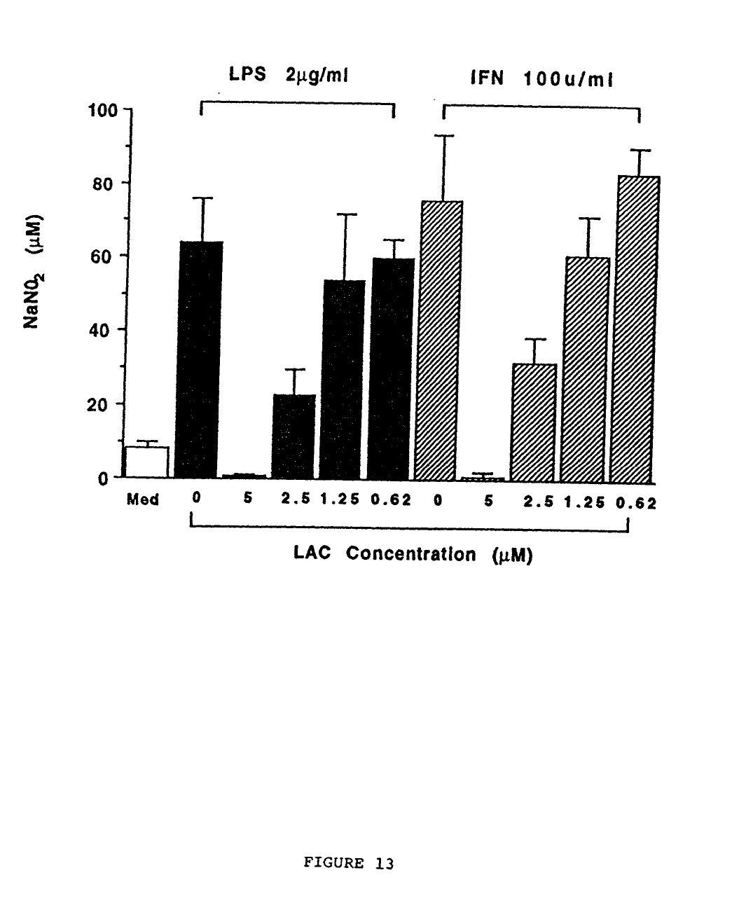

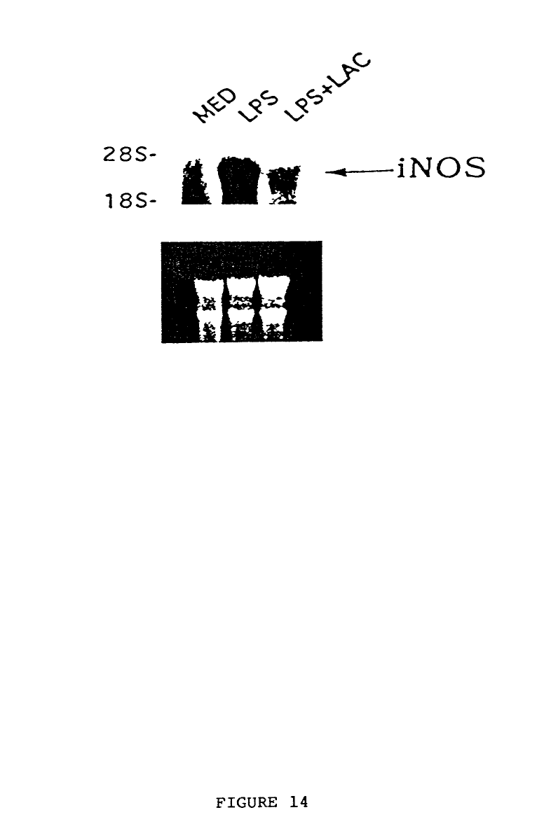

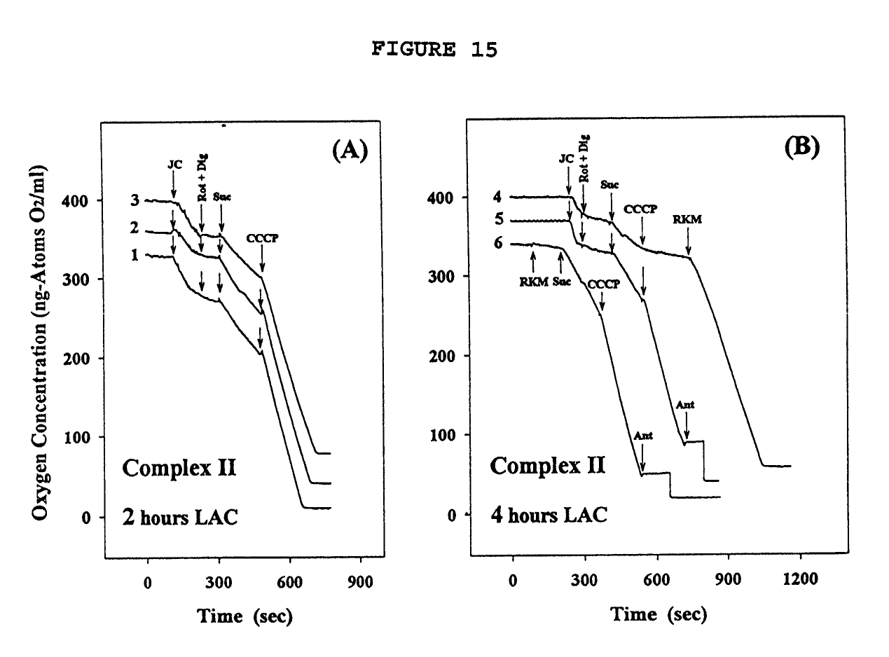

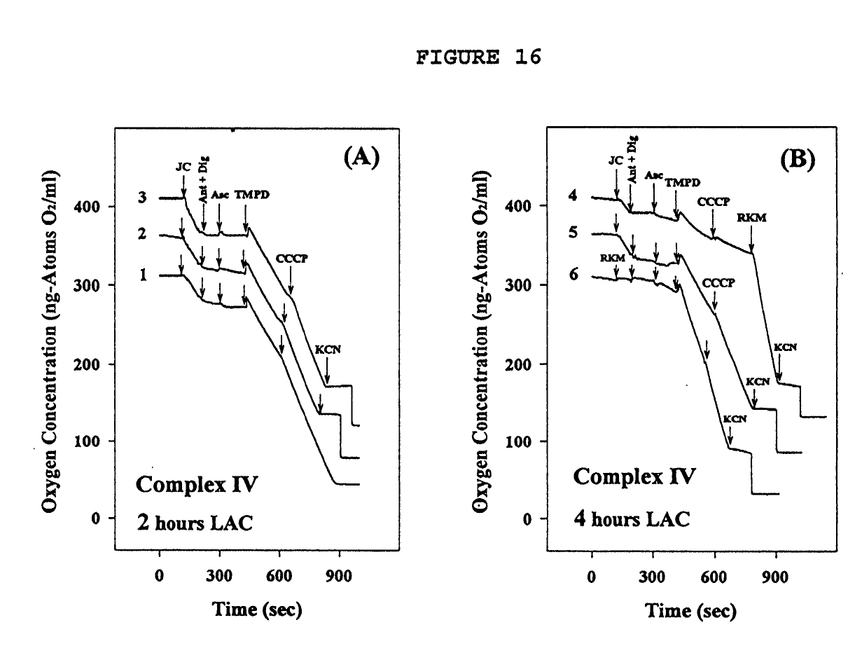

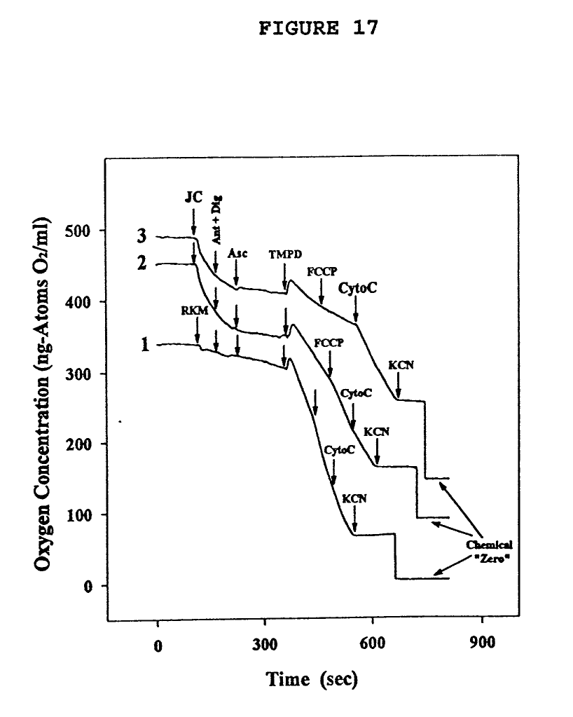

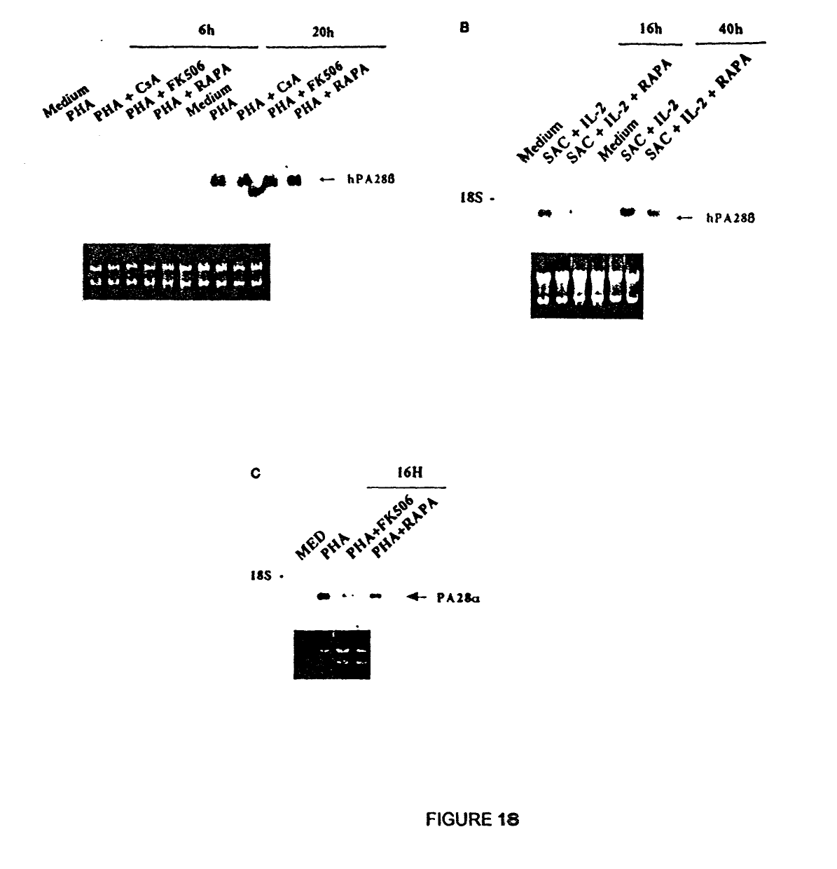

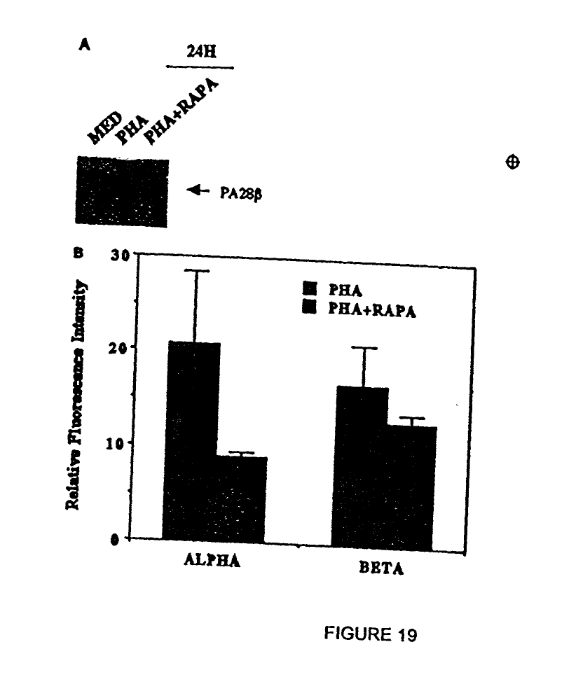

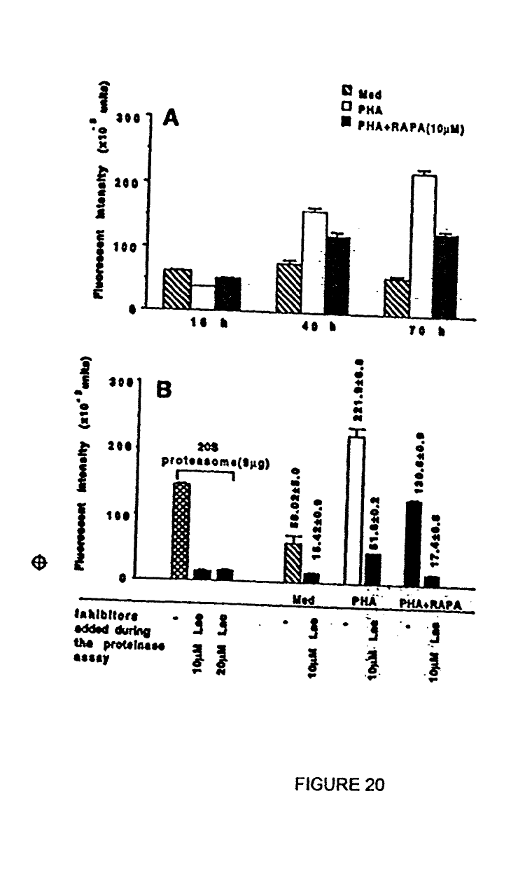

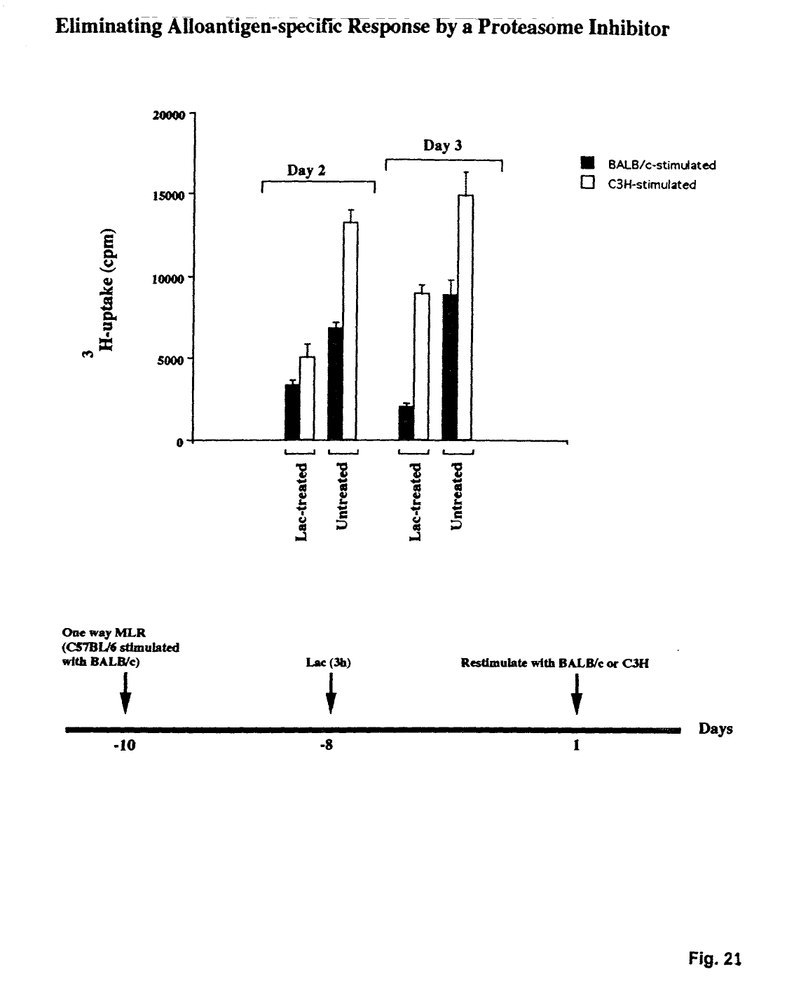

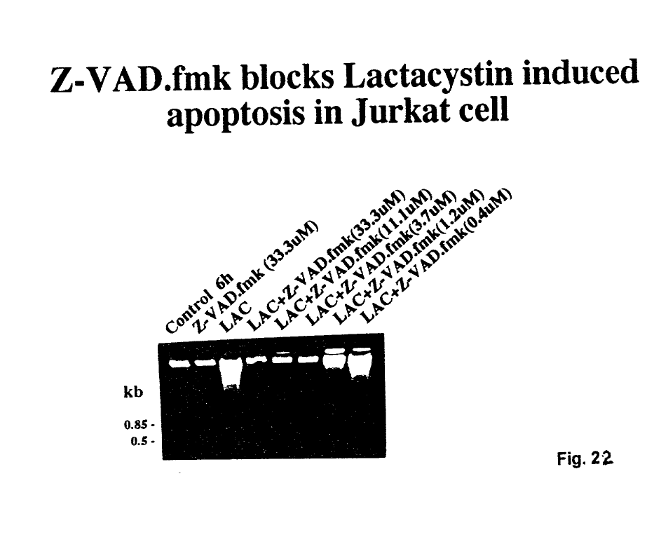

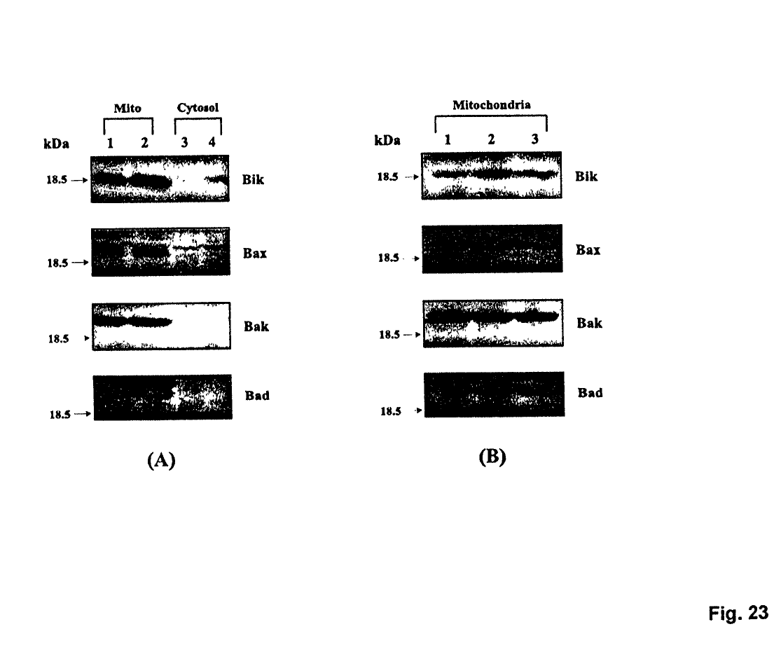

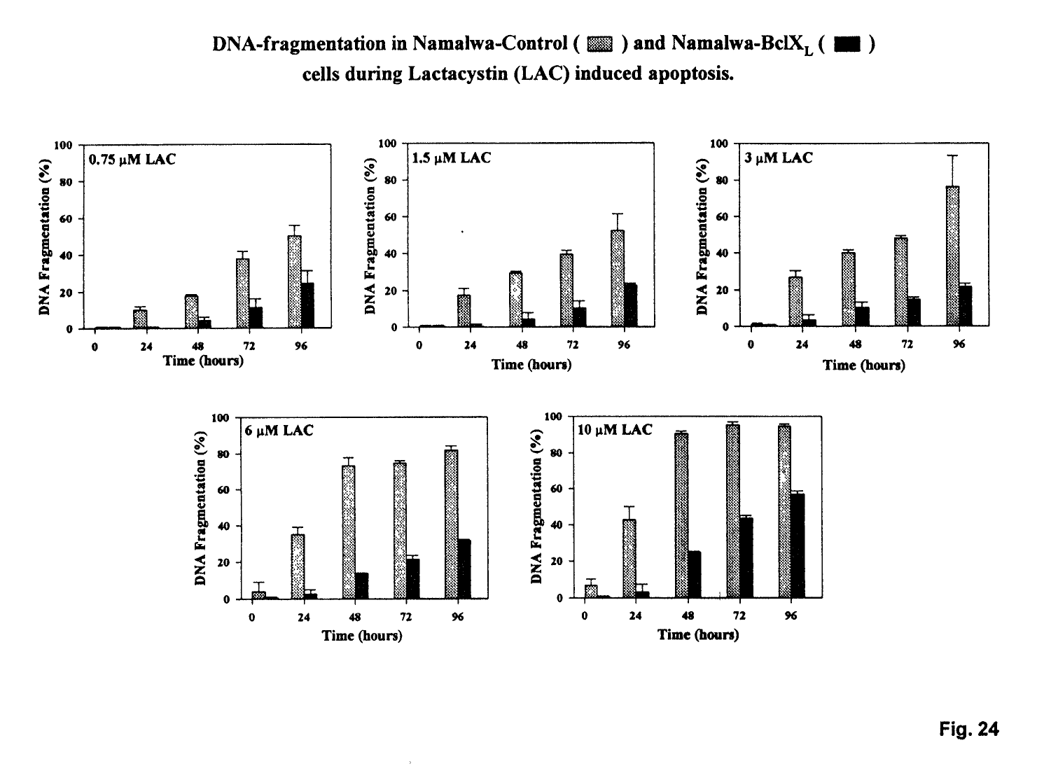

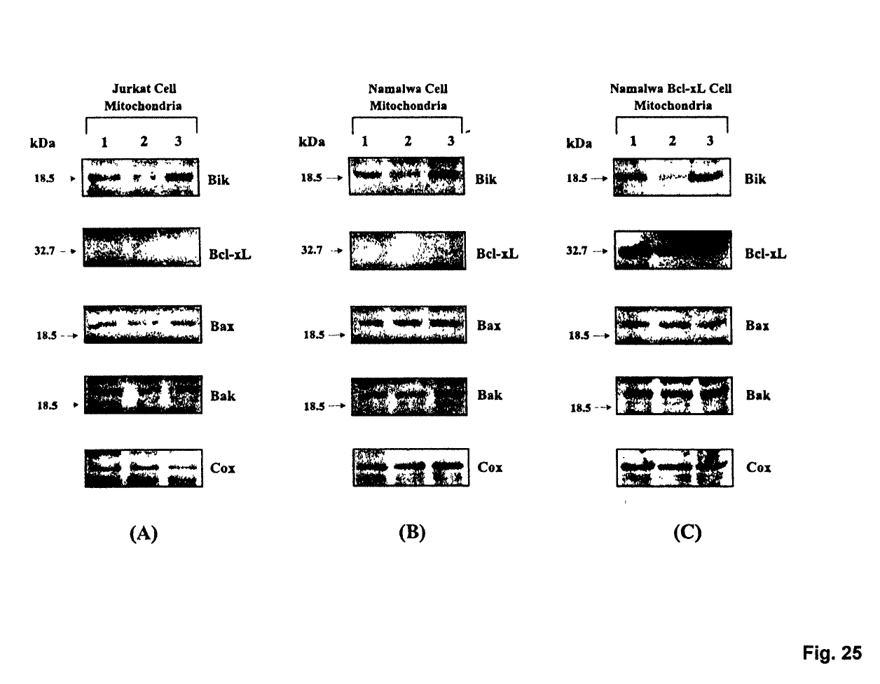

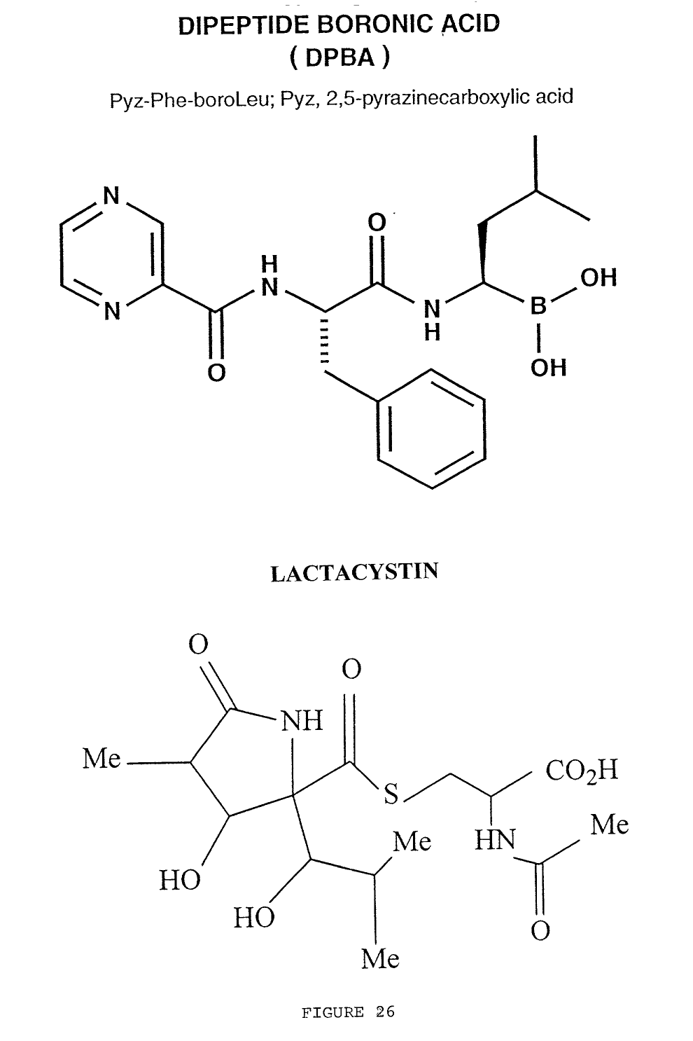

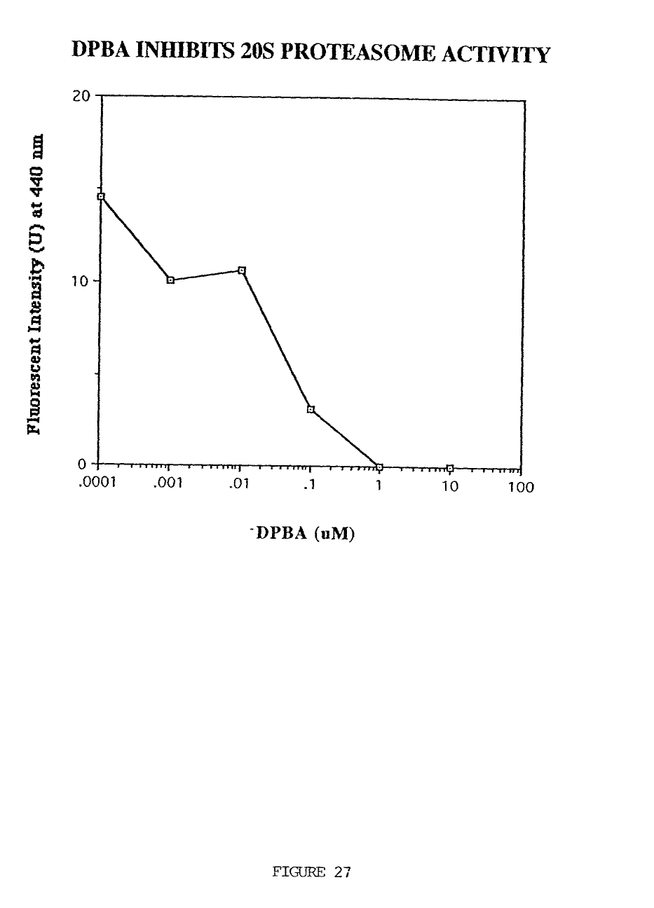

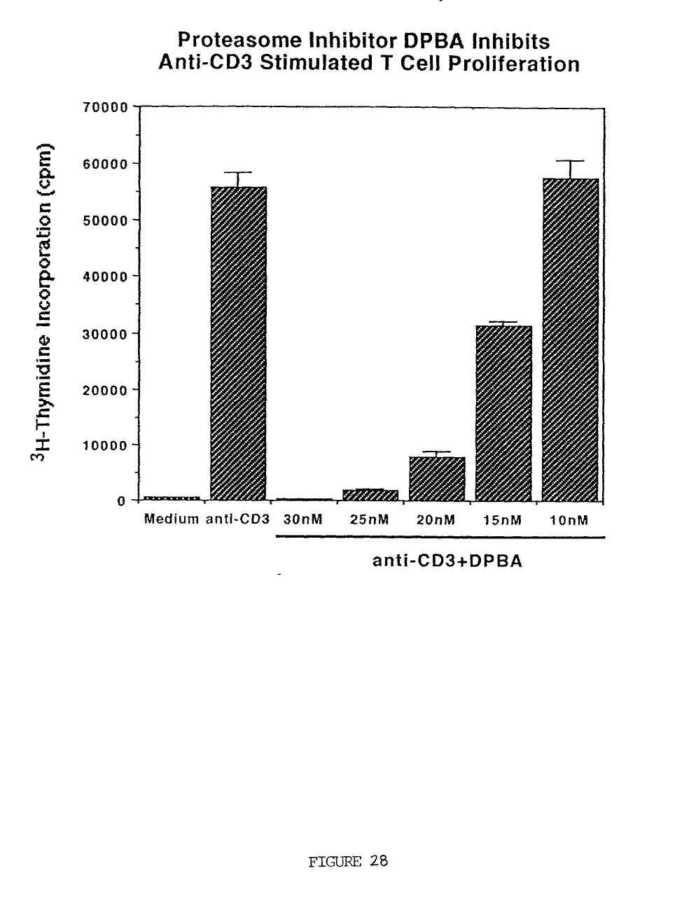

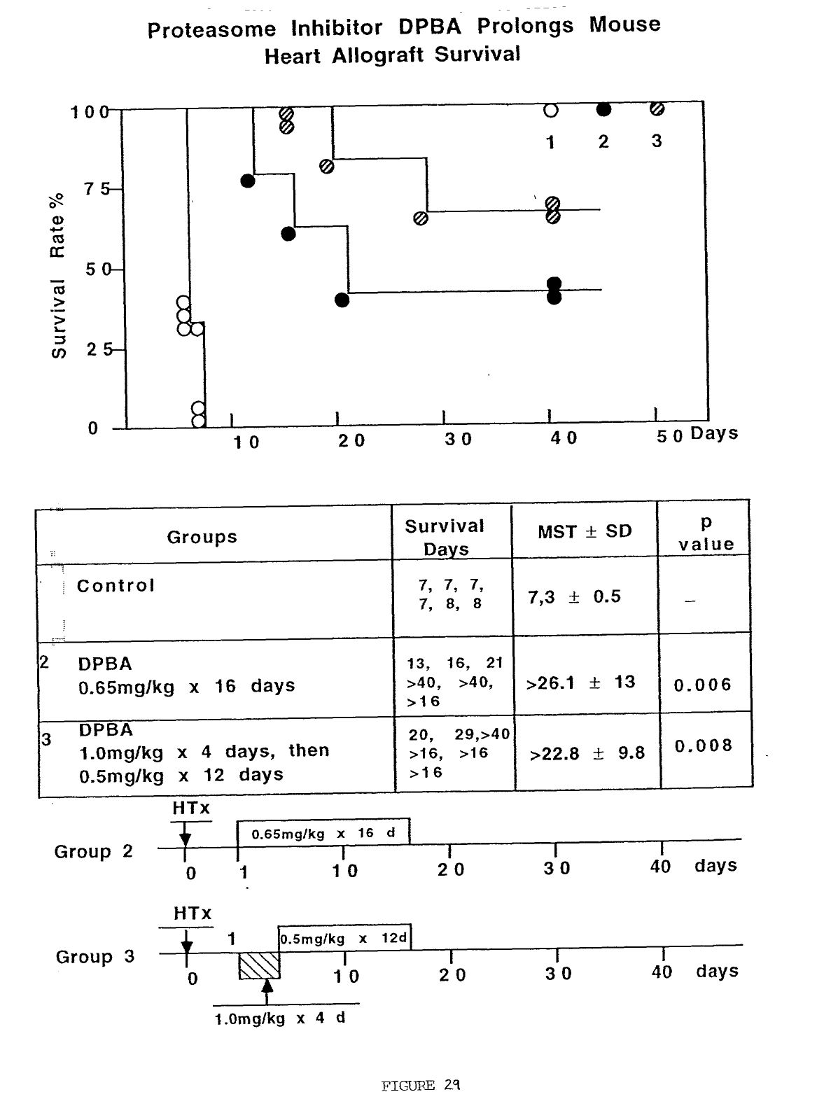

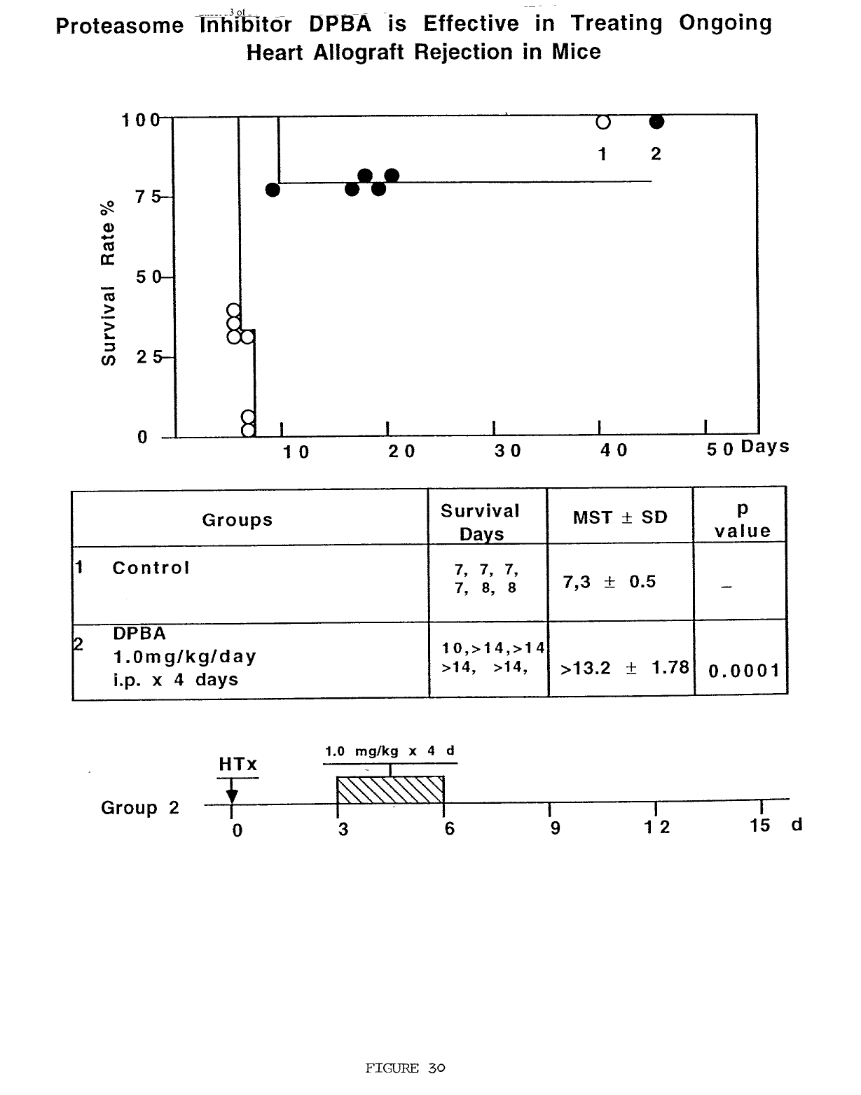

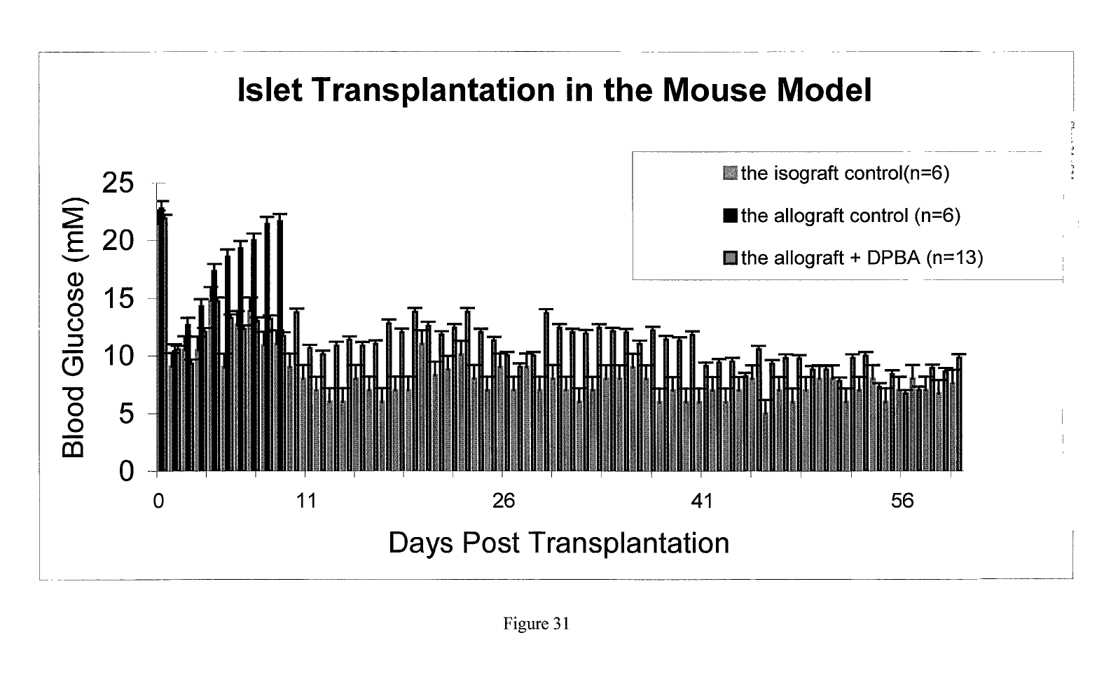

Electron microscopy was also employed to examine the mode of cell death induced by LAC. The resting T cells (cells cultured in medium, FIG. 3A), activated T cells (40 h after PHA activation, FIG. 3C), and Jurkat cells (FIG. 3E) were all healthy looking. Occasional condensed nuclei were observed in medium cultured T cells (FIG. 3A) and this is not unusual. The resting T cells treated with LAC (10 μM) for 24 h were still healthy (FIG. 3B). However, nuclear condensation, which is a hallmark of apoptosis, were frequently observed in activated T cells and Jurkat cells after they were exposed to LAC (10 μM and 6 μM, respectively) for 24 h (FIGS. 3D and F).

Following conclusions are drawn from the results of this section. 1) Resting T cells or T cells in their early activation phase (less that 24 h after PHA-stimulation) are not sensitive to LAC in terms of cell viability. Consequently, there are still a significant percentage of live cells after 64 h culture should LAC be added once at the beginning. 2) Less than 8-9 h of LAC treatment does not affect significantly viability of activated T cells (40 h post PHA activation) or Jurkat cells, according to trypan blue exclusion. 3) Prolonged treatment (24 h) of the activated T cells or Jurkat cells with LAC causes cell death in the form of apoptosis, although signs of apoptosis could be detected as early as 9 h in T cells and 6 h in Jurkat cells after the LAC treatment.

The data in this section further infer following notions. 1) LAC's differential effect on the viability of resting versus cycling cells suggests that it is not simply nonspecific cytotoxicity, but relates to the status of the cell cycle. 2) The cell death without doubt contributes to but cannot solely account for the observed inhibition of proliferation by LAC, since there are still significant percentage ( about 60%) of live cells at the end of the culture according to trypan blue exclusion. Moreover, we will elaborate later that the cell death is a consequence of blockage of cell cycle progress. 3) Admittedly the trypan blue negative cells includes some early apoptotic cells, as evidenced by the fact that DNA laddering could be detected in a largely trypan blue negative population. However, it does not necessarily mean that the whole population is dead. We will later demonstrate that most Jurkat cells treated with LAC for 6 h to 8 h could still progress normally in cell cycle, in spite that a certain degree of apoptosis could be detected in these cells. 4) LAC could be used to study the role of proteasomes in lymphocyte activation and proliferation, as long as the compound is applied only once in the beginning of activation of the resting T cells and the experimentation is carried out in 24 h-40 h, or LAC is present for less than 8 h in the case of cycling cells, since such treatments do not drastically affect the viability of the cells.

A specific embodiment of this invention is the ability of LAC to induce apoptosis mostly in activated and proliferating cells and not in normal resting cells. This has value in eliminating cancerous cells and antigen-specific T cells. The elimination of the latter will create a specific immune tolerance to alloantigens in transplantation, and to selfantigens in autoimmune diseases.

The Effect of LAC is Rapid and Reversible

We next investigated how fast and how long LAC could exert its effects on the lymphocytes, since this information is necessary to assess the requirement of the proteasome activity for events related to cell activation and proliferation. PBMC were pretreated with LAC (10 μM) or medium for a period as indicated in FIG. 4A. The cells were then washed and recultured in the presence of PHA. The thymidine uptake was measured 3 days later. It was clearly demonstrated that 3 h preincubation with LAC was sufficient to cause significant inhibition on the subsequent mitogen-stimulated proliferation in T cells, although 16 h preincubation with LAC was more effective. This result indicates that LAC can enter the cells rapidly within 3 h.

We used Jurkat cells that have high constitutive proteasome activity to evaluate the duration of LAC's effect once the drug entered the cells. Jurkat cells were treated with LAC (6 μM) for 3 h, which was sufficiently long for the compound to enter the cells as shown above. The cells were then thoroughly washed and recuitured, and they were harvested at 0 h, 5 h and 21 h after the wash, and the proteasome activity in the cell lysates was measured using a chromogenic chymotrypsin substrate. We have previously established that the proteinase activity measured by this assay was predominantly (more than 90%) derived from the proteasome (Wang et al., 1997, Eur. J. Immunol., supra). As shown in FIG. 4B, the proteasome activity in Jurkat cells was almost completely inhibited by 3 h preincubation with LAC at 6 μM. Five hours after the LAC was washed out, the proteasome activity in the cells was still significantly inhibited but the inhibition was reduced compared with that at 0 h. By 21 h, the proteasome activity returned to a near-normal level. It is to be noted that the short 3 h treatment with LAC did not affect the viability of the Jurkat cells, and this is also reflected by the normal proteasome activity of the treated cells at 21 h. The result shows that LAC is not stable and loses its activity within 21 h in the cells.

We also investigated whether LAC was stable in the culture supernatant. LAC (6 μM) was added to Jurkat cells culture for 4 h, 6 h, 16 h or 24 h. The conditioned medium was harvested and used to treat fresh Jurkat cells for 3 h, and then the proteasome activity in the lysates of the fresh Jurkat cells was assayed. As shown in FIG. 4C, 4 h to 24 h conditioned media without LAC did not affect the proteasome activity of the fresh Jurkat cells. The media conditioned with LAC up to 6 h could still actively inhibit the enzymatic activity, but after 16 h, the LAC-conditioned media lost their inhibitory effect. The loss of LAC activity in the 16 h and 24 h conditioned medium is unlikely due to trapping of LAC by proteasomes released by dead Jurkat cells, because LAC could rapidly enter the live cells and the equilibrium of the LAC concentration between both sides of the cytoplasmic membrane should be established very fast. Thus, the proteasomes whether released or not should not make a difference in terms of trapping LAC. Besides, we have also noticed that LAC kept in cell free culture medium at 4° C. would lose its activity within 24 h (data not shown). These results indicate that LAC is not only unstable within the cells, but is also unstable in the supernatant.

LAC's capability to enter the cells to inhibit the proteasome activity rapidly (less than 3 h), and its short active duration within the cell and in the culture media (about 16 h) makes the compound a very useful reagent to evaluate the requirement of the proteasome activity in various events during cell activation and proliferation, since we could pinpoint the period when the proteasome activity is critical.

It is an embodiment of this invention, the use of LAC can be regulated in a time course sequence to be most effective at the period when proteasome activity is critical to maximise the effect of LAC on cells.

Proteasome Activity is Required for IL-2Rα Upregulation

In the four systems of T and B cell activation and proliferation studied in the first section, the growth promoting activity of IL-2 is indirectly (for stimulation by PHA, anti-CD3, and anti-CD28 plus ionomycin), or directly (for SAC plus IL-2) involved. We then investigated the role of proteasome in IL-2Rα expression and IL-2 production. As shown in FIG. 5, CD25 was upregulated in CD3⁺ T cells 40 h after stimulation with PHA. When LAC (10 μM) was added in the beginning of the culture, the upregulation was significantly inhibited. On the other hand, IL-2 production by PBMC 2 to 4 days after PHA stimulation in the absence or presence of LAC (10 μM, added at the beginning of the culture) was also examined, but no consistent difference was found (data not shown). Under the experimental condition used, the viability of the LAC-treated cell was reasonable (>80% at 40 h) as described in the previous section as LAC was added only once initially. Moreover, no consistent change of IL-2 production in LAC-treated cells was a functional indication that the cell viability was reasonable and is not of a concern in interpreting the data. The results from this section indicate that IL-2Rα upregulation but not IL-2 production is proteasome-dependent, and the suppressed IL-2Rα expression likely contributes to LAC's inhibitory effect on T cells activation and proliferation. The Proteasome Activity is Critically Required Between G₀ and G₁/S Boundary in T Cells

Like normal T cells, the proliferation of Jurkat cells was also potently inhibited by LAC (data not shown). We used synchronized Jurkat cells to identify the LAC-sensitive phase(s) of the cell cycle. Jurkat cells were first synchronized at the G2/M boundary by nocodazole (FIG. 6A). The cells were released from the blockage by washing out nocodazole. In the control sample, more than half the cells traversed through the M phase and arrived at the G: phase within 4 h. In the test sample, LAC (6 μM) was added to the culture 3 h before the release, so the compound could have enough time to enter the cells. LAC was also added to the culture after the release. However, the Jurkat treated with LAC traversed through the M phase to the G₁ phase at a similar pace as the control cells. Since the total duration of the assay was around 7 h (3 h preincubation plus 4 h after the release), LAC was certainly active during this period. The fact that most of synchronized Jurkat cells could traverse through G2/M to G1 in the presence of LAC for 7 h again suggests that the viability of the cells thus treated is not a matter of concern. This result shows that the G₂ to G₁ progression is not proteasome-dependent.

We next studied requirement of the proteasome activity for the progression from the G₁/S boundary to the G₂/M phase. The Jurkat cells were synchronized at the G₁/S boundary by HU blockage. The cells were then released by washing out HU. Within 9-12 h, the majority of the cells progressed to the S and G₂/M phase (FIG. 6B). When LAC was added to the culture immediately after the release, it slowed but did not block the cell cycle progression from the G₁/S boundary to the G₂/M phase, as evidenced by the histograms at 6 h and 9 h post the release. It is to be noted that although the percentage of cells in the S/G2/M phase in the LAC-treated sample was similar to that of controls (the inset table of FIG. 6B), the peak of fluorescence was lagged behind (histogram array). Beyond 9 h, the cells gradually lost their synchronization, the viability of the cells started to decline and LAC gradually lost its activity, so the data became difficult to interpret. The result from this part suggests that the proteasome activity is required for optimal progression from the G₁/S boundary to the G₂/M phase, because the progression could still proceed albeit at a slower pace when the proteasome activity is inhibited. The result also implies that the absolutely proteasome-dependent window during the cell cycle, as evidenced by the near-total inhibition of S phase entry in LAC-treated mitogen-stimulated lymphocytes according to the proliferation data, must be in the G1 phase before the target point of HU, which inhibits ribonucleotide reductase in the G./S boundary (Brown et al., 1996, Cell 86:517).

The cycling Jurkat cells are obviously not the best model to study the events in the G₁ phase since the G₂/M synchronization become desynchronized by the time the cells re-enter the S phase, and there is no appropriate method to synchronize the Jurkat cells at the early G₁ phase. We therefore decided to use mitogen-stimulated normal T cells to study the role of the proteasome in the G₁ phase.

T cells from PBMC were at Go when isolated. After 16 h stimulation with PHA, they remained before the S phase (FIG. 6C). At 40 h, about 20% of the cells were in the S and G₂/M phases. The peak of ³H-thymidine uptake according to a 16 h pulse was between 48 h and 64 h (data not shown), although at 64 h, the cells in the S and G₂/M phases were still about 20% (FIG. 6C). The lack of an increase in percentage of cells in the S and G₂/M phases at 64 h compared with that at 40 h was likely due to the exit of the cells from the S and G₂/M phase. It is to be noted that the cycling T cells in this system never reaches 100%, because about 15% of the cells were non T cells, and an additional 20% were non responsive T cells. Taken the cell proliferation and cell cycle analysis together, the G₁/S boundary of the cycling T cells should be between about 35 h and 48 h after the PHA stimulation. The boundary was broad because the synchronization was not ideal.

In this model, the role of the proteasome in the S phase entry was examined. As shown in FIG. 6C, LAC added once at 16 h could totally block the S phase entry when examined at 40 h. We have noticed that when the cell viability was evaluated at 40 h, there was an increase of cell death comparing the 16 h addition of LAC with the 0 h addition (about 25% vs about 17%, data not shown). The increased cell death was also reflected in the cells with <2N DNA in the 40 h histogram. However, such a viability was still reasonable and would not invalidate our conclusion. According to ³H-thymidine uptake, LAC was strongly inhibitory even added as late as 40 h (FIG. 6D). However, no difference on the percentage of the population in the S and G₂/M phase was observed at 64 h whether or not LAC was added at 40 h according to flow cytometry (FIG. 6C). The discrepancy could be explained by the fact that the 20% cells were already in the S and G₂/M phases at 40 h when LAC was added. LAC prevented additional cells from entering into the S phase, therefore the lack ³H-thymidine uptake. At the same time, the drug slowed the cell cycle progression from the G₁/S boundary to the G₂/M phase, hence the lingering population in the S and G₂/M phases according to flow cytometry.

It is worth mentioning the inhibition of proliferation by LAC was a combinatory effect of cell cycle progress and cell death, the latter possible being the consequence of the former. The later the compound was added when more T cells are activated, a larger proportion of the effect should be attributed to cell death caused by LAC. The extensive cell death for the sample treated with LAC at 40 h was not fully reflected in the flow cytometry (FIG. 6C) as cells with less than 2N DNA. This was due to that the histogram was gated on a region of largely viable cells.

The results from this section indicate that the proteasome activity is not required from the G₂/M to the G₁ phase. It optimizes the progression from the G₁/S boundary (as defined by the hydroxyurea target point) to the G₂/M phases, and it is absolutely required for the progression from the Go to the S phase.

In a specific embodiment of this invention LAC is used to reverse ongoing graft rejection during a rejection episode. Most immunosuppressive drugs do not have the capability to reverse rejection once it begun. The use of LAC overcomes the prior art.

The Proteasome Activity is Essential for CDK2 but not for CDK4 Function

Cyclin-dependent kinases (CDK) are critical for cell proliferation. CDK4 is essential in the early to mid-G₁ phase to facilitate the S phase entry (Tam et al., 1994, Oncogene 9:2663; Lukas et al., 1995, Oncogene 10:2125) and CDK2 is critical in the late G₁ as well as throughout the S phase for the cell cycle progression (Van der Heuvel et al., 1993, Science 262:2050). We therefore examined the role of the proteasome in CDK4 and CDK2 activities in mitogen-stimulated T cells. In all the models used in this section, LAC was added only once at the beginning of the culture. Consequently, the viability of the LAC-treated cells was good for the first 16 h and was reasonable at 40 h, and was not a factor that might interfere with the interpretation of the results.

As shown in FIG. 7A, the resting T cells had some CDK4 activity, and the activity reached a plateau within 16 h of the activation. This was in agreement with the critical role of CDK4 in the early G phase. Inhibition of the proteasome activity by LAC from 0-16 h (LAC added once at 0 h) did not affect the CDK4 activity when examined at 16 h and 40 h (FIG. 7A). This indicates that the induction and maintenance of CDK4 activity during the G1 phase is not proteasome-dependent.

In contrast to CDK4, the CDK2 activity was augmented at 16 h but the augmentation was more prominent at a later stage close to 40 h after the mitogen-stimulation (FIG. 7B), and this reflected its essential role starting from the late G₁ phase and extending to the early S phase. The presence of LAC from 0 h to 16 h (LAC added once at 0 h) significantly inhibited CDK2 activity at 16 h and more so at 40 h. Therefore, the proteasome activity during the early activation stage (0 h-16 h) is essential for the activation of the kinase at the G1 phase and early S phase. The unchanged CDK4 activity in the LAC-treated cells at 40 h served as an internal control for the repressed CDK2 activity and indicating the latter was not due to the viability problem.

Since at the late G₁ phase Cyclin E is the predominant partner of CDK2 (Sherr, 1993, Cell 73:1059), we next examined the Cyclin E-associated CDK activity. As shown in FIG. 7C, in spite that the Cyclin E protein was increased after the LAC treatment (LAC added once at 0 h), the Cyclin E-associated kinase activity was almost completely inhibited by LAC. These results indicate that the CDK2 activity, and most likely the Cyclin E-associated CDK2 activity in the late G₁ phase is proteasome-dependent. The results also suggest that the inhibition of the CDK2 activity is probably an important mechanism accountable for the LAC's effect in blocking the S phase entry.

It is an embodiment of this invention to have elucidated a downstream target for proteasome activity. That is CDK2, more specifically Cyclin E-associated CDK2 activity. It is also provided that with this knowledge, inhibitors of CDK2 can be used alone or in combination with proteasome inhibitors. It is further provided that the aforementioned compositions are of a pharmaceutically effective amount to induce apoptosis or for any other cellular or physiological effect. Since CDK4 activity is important in G₀ to G₁, progression and it is not affected by proteasome activity, it is conceivable that inhibitors for CDK4 can be used in combination with proteasome inhibitors of a pharamceutically effective amount to achieve additive effect in blocking cell proliferation and in any other relevant cell function.

Inhibitors in this application are defined as any element capable of silencing the activity of a protein at the level of gene transcription, translation, or post-translational modification of the protein as well as elements capable of interfering with the protein. These may include but are not limited to antibody or other ligands, anti-sense or antagonist molecules.

Degradation of Cyclin E but not Cyclin A is Proteasome-Dependent

It is a specific embodiment of this invention that contacting LAC with CDK2 is inhibitory to CDK2 activity, more particularity it is the inhibitory effect of LAC on Cyclin E. The inhibitory effect of LAC is the disruption of cell cycling.

Oscillation of cyclins during the cell cycle is a mode of regulation for the CDK activities. Since the CDK2 activity is proteasome-dependent, and CDK2 associates predominantly with Cyclin E and cyclin A at the G₁/S boundary and during the S phase respectively (Pagano et al., 1992, EMBO J. 11:961; Hall et al., 1995, Oncogene 11:1581), we studied the role of the proteasome in degradation of these two cyclins. As shown in FIG. 8A, the Cyclin E level was apparently increased around 40 h after PHA stimulation of the T cells, which were then at the G₁/S boundary. If the activated cells were treated with HU, the Cyclin E level was significantly enhanced comparing with those treated with PHA alone (FIG. 8A). This reflects a better synchronization at the G₁/S boundary by HU, and was consistent with our knowledge that the Cyclin E level peaked at the boundary. After the boundary, the Cyclin E level started to decline, and the decline was prevented by LAC (FIG. 8A). This clearly demonstrates that the degradation of Cyclin E is a proteasome-dependent process, although whether the increased Cyclin E level contributes to LAC's effect on the cell cycle is a matter of debate.

For cyclin A, the level was increased around the late G₁ phase after the mitogen stimulation as shown in FIG. 8B. The blockage of the cycle at the G₁/S boundary with hydroxyurea did not further increase the cyclin A level. However, when the cycle passed the boundary and entered the S phase, the cyclin A level was significantly augmented (FIG. 8B), consistent with the notion that cyclin A is mainly an S phase cyclin. Unlike that of Cyclin E, the level of cyclin A did not decline during the S phase and LAC did not affect the level during this period. This suggests that the proteasome is not involved in cyclin A degradation, at least in the G₁ and S phases, and that LAC's effect on inhibiting cell proliferation is unlikely mediated via the cyclin A levels. The G₁/S phase synchronized T cells represented activated cells, and prolonged exposure to LAC would cause significant cell death. However, 6 h treatment of LAC did not apparently affect the cell viability, while the blockage of Cyclin E degradation but not cyclin A degradation was obvious at that time point. Moreover, cyclin A could be considered as an internal control for Cyclin E indicating that the LAC-induced cell death should not affect the conclusion in this section.

The Role of Proteasome in Regulating Levels of CDK Inhibitors p27^{Kip1} and p₂₁^{Cip1}

In a specific embodiment, LAC is capable of suppressing the up regulaion of the CDK inhibitor p21^{Cip1} and in blocking the degradation of the CDK inhibitor p27^{Kip1}.

In addition to the cyclin levels, the CDK activities are also controlled by several low molecular weight inhibitors. We have examined in this study the effect of the proteasome on the CDK inhibitors p27^{Kip1} (Hall et al., 1995, supra) and p21^{Cip1} (el-Deiry et al., 1993, Cell 75:817). As shown in FIG. 9A, the resting T cells had a high level of P27^{Kip1} and the level decreased gradually when the cells moved to the G₁/S boundary 40 h after the mitogen-stimulation. This is in agreement with previous reports (Hengst et al., 1996, Science 271:1861; Nourse et al., 1994, Nature 372:570). The presence of LAC (added once at 0 h) significantly blocked the decrease when assayed at 16 h, showing that the degradation is a proteasome-dependent process. The blockage was less obvious when assayed at 40 h, probably because the gradual loss of LAC activity during the 40 h culture. The result suggests that the blocking of p27^{Kip1} degradation is a contributing mechanism contributing for the inhibitory effect of LAC on the CDK2 activity. Unlike p27^{Kip}, p21^{Cip1} had a low level of expression in resting T cells. The level was rapidly augmented after 16 h PHA activation, and the high level was maintained at the G₁/S boundary at 40 h (FIG. 9B). Such an induction suggests that p21^{Cip1} might be required in the G phase for roles other than a CDK inhibitor. Interestingly, LAC strongly suppressed the upregulation of p21^{Cip1} in the G₁ phase, indicating that the expression of p21^{Cip1} is proteasome-dependent, and suggesting that the proteasome might facilitate cell proliferation via its role in p21^{Cip1} upregulation during the G₁ phase. In this experiment, LAC was only added once at the beginning of the culture, and the viability of the treated cells at 16 h was good (83˜) and should not be a concern in drawing the conclusion.

Disruption of Cell-Cell Interaction

Cell-cell interaction is essential in antigen presentation and in T cell's help to T and B cells. The adhesion molecules are necessary to establish the cell-cell interaction. Blocking the adhesion molecules ICAM-1 and LFA-1 is known to inhibit immune responses and to suppress graft rejection. Our data clearly shows that inhibition of the proteasome activity will effectively interfere with the cell-cell interaction during lymphocyte activation in both human (FIG. 10) and mouse (FIG. 11) systems, and the upregulation of an adhesion molecule ICAM-1 is repressed by the proteasome inhibitor lactacystin (FIG. 12). Therefore, inhibition of the proteasome activity will be a useful way to control undesirable immune responses during graft rejection, autoimmune diseases, and inflammation.

Proteasome Activity is Required for Nitric Oxide Production

Nitric oxide (NO) produced by macrophages is involved in inflammation and septic shock. We have shown that inhibition of the proteasome activity could effectively repress the endotoxin LPS-induced NO production (FIG. 13). The usefulness of proteasome inhibitors in inflammation and in septic shock is implicated. FIG. 14 demonstrates that proteasome activity is required for NO synthase expression. The addition of LAC decreases the expression of mRNA for NO synthase.

The Effect of Proteasome on Mitochondrial Function

Mitochondria are pivotal organelles in the cells and their primary function is to produce ATP via the Krebs cycle coupled to the oxidative phosphorylation of the respiratory chain. An intact function of mitochondria is also required for proper cell viability. Damage of the mitochondrial membrane potential or release of cytochrome C or other apoptogenic factors from the mitochondria to the cytosol will induce cell death via apoptosis.

In our study, we have found that the electron transport in mitochondria of Jurkat T lymphocytes is dependent on the intact activity of the proteasome. A proteasome-specific inhibitor lactacystin (LAC) could rapidly (within 4 h) reduce the electron transport at the complex IV of the respiratory chain, and the effect could be reversed by adding back exogenous cytochrome C (cytoC).

In FIG. 15, the respiration of Jurkat cells treated with LAC for 4 h (curve 4) but not for 2 h (curve 3) could not be resumed by adding succinate after Complex I blockage, and CCCP failed further to stimulate the respiration as it could in control Jurkat cells and in rat mitochondrial preparation (curves 5 to 6, respectively). Adding rat kidney mitochondria to the blocked reaction results in normal respiration (curve 4), showing the reagents and the oxygen electrode are functional. The results indicate that LAC compromises the electron transport after Complex I.

In FIG. 16, Jurkat cells treated with LAC for 2 h (curve 3) had similar O₂ consumption after Complex III, like that of untreated Jurkat cells (curve 2) and rat kidney mitochondria (curve 1). After 4 h LAC treatment, the O₂ consumption of the Jurkat cells could not be resumed by ascorbate and TMPD to a level similarly high as that of untreated Jurkat and rat mitochondria, and the decoupling reagent CCCP had no effect in the treated cells (curve 4). Adding back rat kidney mitochondria into the assay could resume the O₂ consumption, showing a functional assay system. Curves 5 to 6 are untreated Jurkat cells and rat kidney mitochondria, respectively, showing normal function of Complex IV. This result shows that the LAC treatment caused compromised function in the electron transport at Complex IV.

In FIG. 17, Jurkat cells treated with LAC (curve 3) have reduced augmentation of O₂ consumption after the addition of ascorbate and TMPD, compared with untreated Jurkat cells (curve 2) and rat kidney mitochondria (curve 1). FCCP could not further stimulate the respiration, as it could in normal Jurkat cells and rat kidney mitochondria. When exogenous cytochrome c (CytoC) was added to the LAC-treated cells, the respiration resumed to a rate similar to that of untreated Jurkat cells and mitochondria. CytoC had no additive effect in stimulating respiration in normal Jurkat cells and rat mitochondria (curves 2 and 3, respectively).

The implication of aforementioned findings is as follows:

In hyperthyroidism, the mitochondrial activiy is overactive due to the effect of the thyroid hormone. This results in many symptoms such as excessive body heat, and imbalance of energy uptake and consumption. The proteasome inhibitors could reduce the rate of mitochondrial respiration and have therapeutic effect to this disease.

In fast-growing cells such as cancer cells or activated lymphocytes, the mitochndria are more active than in normal cells in order to meet the energy requirement of a high metabolic activity of these cells. Consequently, inhibition of the mitochondrial respiration could curb the proliferation of the cancer cells or activated lymphocytes while have less detrimental effects to normal resting cells. In addition, apoptosis could be induced in the cycling cells but not resting cells. Thus, inhibition of the proteasome activity will have therapeutic effect in cancer and in diseases involving lymphocyte activation and proliferation, such as seen in graft rejection and autoimmune diseases.

Rapid Assays for A High Through-Put Screening Procedure to Identify Additional Proteasome Inhibitors

In our study, we have shown that about 70-80% of the chymotrypsin-like activity in the lymphocyte lysates is derived from the proteasome (FIG. 20). In a positive control, LAC at 10 μM could inhibit 90% of the 20S proteasome activity which was in a range similar to that of the cell lystates. Increasing the concentration of LAC to 20 μM did not further increase the inhibitory effect, suggesting that the LAC concentration used was already saturating. The remaining 10% activity might be derived from non-proteasome proteinases in the 20S proteasome preparation. When 10 μM LAC was added to the 70-h cell lysate, it inhibited 73.4%, 76.7% and 86.7% of total chymotrypsin-like activity in the lysates from medium-, PHA- and PHA plus RAPA-treated PBMC, respectively, and those percentages represented the portion of enzymatic activity from the proteasome.

The implication of this finding is that mammalian cell lysates without other purification could be used as a convenient source of proteasomes. Tagged substrates specific for the known proteasome activities, such trypsin-like, chymotrypsin-like, and PGPN activities can be used as displaying parameters. Known compounds could be added into this enzyme/substrate system, and the compound(s) that inhibit(s) one or several aforementioned enzyme activities of the lysate above a certain threshold (for example 40%) will be identified as proteasome inhibitors. These assays could be modified to use purified or partially purified 20S or 26S proteasome as a source of the proteasome enzymes. Since such assays are simple (only three components) and rapid (only several minutes of reaction period), they could be adapted for high through-put screenings, and included in a kit format.

The Effect of Immunosupressive Drugs on Proteasome Function

Rapamycin (RAPA) is a potent immunosuppressive drug, and certain of its direct or indirect targets might be of vital importance to the regulation of an immune response. Seven RAPA-sensitive genes are known and one of them encoded a protein with high homology to the α subunit of a proteasome activator (PA28α). This gene was later found to code for the β subunit of the proteasome activator (PA28β). Activated T and B cells had upregulated PA28β expression at the mRNA level. Such upregulation could be suppressed by RAPA, FK506, and cyclosporin A (CsA). RAPA and FK506 also repressed the upregulated PA28α messages in PHA-stimulated T cells. At the protein level, RAPA inhibited PA28α and PA28β in the activated T cells according to immunoblotting and confocal microscopy. Probably as a consequence, there was a fourfold increase of proteasome activities in the PBMC lysate after the PHA activation. RAPA could inhibit the enhanced part of the proteasome activity. Considering the critical role played by the proteasome in degrading regulatory proteins, a proteasome activator is a relevant and important downstream target of rapamycin, and that the immune response could be modulated through the activity of the proteasome.

A lot of efforts have been made to identify direct targets of RAPA. It is now known that RAPA complexes with a 12 KD FK506-binding protein (FKBP12) (Harding et al., 1989, Nature 341:371; Siekierka et al., 1989, Nature 341:755). The RAPA-FKBP12 complex then binds to cytoplasmic proteins termed TOR1 and TOR2 (target of rapamycin) in yeast (Kunz et al., 1993, Cell 73:585; Helliwell et al., 1994, Mol. Biol. Cell. 5:105), and FRAP and RAFT1 in mammalian cells (Brown et al., 1994, Nature 369:756). These target proteins have high degree of homology in their primary sequences, and their C-terminal sequences share certain homology with catalytic domains of both PI-3 kinase and PI-4 kinase.

The mRNA expression of most genes so far studied, whether they are constitutively expressed or induced after stimulation, are not sensitive to RAPA (Tocci et al., 1989, J. Immunol. 143:718; Shan et al., 1994, Int. Immunol. 6:739). It follows that the genes that are sensitive to RAPA at the mRNA level have a good probability of being secondary targets of RAPA and being pivotal in controlling the immune response. Expression of PA28β at mRNA and protein levels was found to be sensitive to RAPA, so was that of the PA28α subunit which shares a high degree homology with PA28β. It was found that proteasome activity was repressed by the drug.

In HeLa cells, PA28β expression was dramatically upregulated at the mRNA level by IFNγ treatment after 24 h. This was similar to the regulation of PA28α (Realini et al., 1994, supra). When human tonsillar T cells were stimulated by PHA, the PA28β expression was augmented after 20 h, and the augmentation could be suppressed by 10 nM RAPA as expected (FIG. 18A). In addition, the expression was also sensitive to CsA (1 μM) and FK506 (10 nM). In tonsillar B cells, SAC and IL-2 upregulated the PA28β mRNA expression, and RAPA was inhibitory (FIG. 18B). Similarly, the mRNA expression of PA28α, which has a high degree of homology with PA28β, was upregulated in PHA-activated T cells, and the upregulation was repressed by FK506 and RAPA (FIG. 18C).

Expression of PA28β and PA28α at the protein level was also examined. The result of immunoblotting demonstrated that the activated T cells had increased PA28β compared with resting T cells, and the increase was inhibited in the presence of RAPA (FIG. 19A). Since the anti-PA28α antiserum did not seem to recognize the denatured proteins, we used confocal immunofluorescent microscopy to examine the PA28α protein as well as the PA28β protein in the T cells. The experiment was carried out in an one-way blind fashion, the microscopy operator without being informed of the treatment of the cells. As shown in FIG. 19B, RAPA plus PHA-treated T cells had significantly lower levels of both PA28α and PA28β proteins compared with T cells treated with PHA alone. We have noticed that although the difference between the PHA-activated T cells in the absence and presence of RAPA was highly significant (p<0.0001), the difference of the numeric values of the mean fluorescence intensity between the two types of cells, especially in the case of PA28β, was rather small. However, there was a high standard deviation in the PHA-treated samples. A closer inspection revealed that about 40% of the cells treated with PHA alone had elevated PA28β and PA28α signals while the rest had basal level expression. This caused the high standard deviation. Considering that there were 20% non T cells in the T cell preparation, and that PHA does not activate all the T cells in the culture simultaneously, those 40% cells with the high signals probably represented the truly activated T cells. Therefore, the actual difference between the activated and drug-repressed cells could be much bigger than the data presented in the histogram.

Taken together, our data indicates that RAPA inhibits the expression of PA28α and PA28β at both mRNA and protein levels. The inhibition of the PA28 mRNAs is a likely cause for the observed decrease of the corresponding proteins. However, we could not exclude the possibility that RAPA might also act directly at the translation level for PA28α and PA28β.

In as much as PHA could upregulate and RAPA could repress expression of the proteasome activator PA28β and PA28α in the T cells, it is logical to examine changes of proteasome activity in these cells. PBMC lysates were assayed for their proteinase activity at pH 8.2 which favors the proteasome activity, using a chymotrypsin substrate as a representative parameter. Forty and seventy hours after stimulation by a T cell mitogen PHA, the chymotrypsin-like activity in the PBMC increased 2.1 fold and 3.8 fold, respectively (FIG. 20A). RAPA at 10 nM repressed 23.1% and 41.1% the activity in the PBMC, respectively, at these time points.

We then tried to determine the part of enzyme activity in the lysates conferred by the proteasome. In a positive control, LAC at 10 μM could inhibit 90% of the 20S proteasome activity which was in the range similar to that of the cell lysates (FIG. 20B). Increasing the concentration of LAC to 20 μM did not further increase the inhibitory effect, suggesting that the LAC concentration used was already saturating. The remaining 10% activity might be derived from non-proteasome proteinases in the 20S proteasome preparation. When 10 μM LAC was added to the 70 h cell lysate, it inhibited 73.4%, 76.7% and 86.7% of total chymotrypsin-like activity in the lysates from medium-, PHA- and PHA plus RAPA-treated PBMC, respectively, and those percentages represented the portion of enzymatic activity from the proteasome (FIG. 20B). The net proteasome activity increased by 4 fold from 42.6×10³ units/20 μg protein in unstimulated cells to 170.3×10³ units/20 μg protein in the PHA-activated cells. In RAPA-treated cells, the activity decreased to 113.2×10³ units/20 μg protein. This equated to 33.6% inhibition of the total activity, or 44.7% of the augmented proteasome activity in the PHA-treated PBMC. It is therefore demonstrated that RAPA could inhibit the enhanced proteasome activity during T cell activation.

It is an embodiment of this invention to have identified known immunosupressive drugs including rapamycin, FK506 and cyclosporin A as inhibitors of enhanced proteasome activity. It is therefore a specific embodiment of this invention for providing these immunosupressive drugs of a pharmaceutically effective amount and in combination with specific proteasome inhibitors of a pharmaceutically effective amount, as an example but not limited to LAC or its analogues to achieve an additive effect in blocking cell proliferation and any other relevant cell function. Such combinations as described can be used but are not limited to the treatment of cancer, graft rejection and autoimmune diseases.

Elimination of Alloantigen-Specific Response

The results of the functional assay shown in FIG. 21 suggests, that there is clonal deletion of BALB/c-specific T cells when proteasome activity of alloantigen-activated T cells are inhibited for a brief period. The consequences of this finding suggests that proteasome inhibitors can be administered when specific T cells are activated, thereby potentially eliminating the activity of specifically activated T cells while leaving non-activated T cells intact. It is therefore an embodiment of this invention to use proteasome inhibitors, particularly lactacystin in transplantation and autoimmune diseases where certain undesirable activated T cells can be repressed or eliminated and the rest of the T cell population is generally unaffected by such inhibitors.

The Effect of Caspase Inhibitor zVAD.fmk. on LAC-induced DNA Fragmentation

The effect of lactacystin as an apoptotic agent in Jurkat cells is shown in FIG. 22, by the typical apoptotic sign of DNA laddering. Addition of the broad spectrum caspase inhibitor zVAD.fms demonstrated an inhibitory effect on DNA fragmentation that is concentration responsive. This result indicates that the lactacystin-induced apoptosis in Jurkat cells is caspase-dependent.

The Effect of Lactacystin on a Pro-Apoptotic Bcl-2 Family Member, Bik

The results shown in FIG. 23 panel A, show that Bik, Bax, Bak, and Bad are predominantly located in the mitochondrial fraction. Treatment with lactacystin does not appear to have altered the amounts of Bax, Bak and Bad (FIG. 23 panels A and B). There is however a demonstable increase in the amount of Bik in the lactacystin treated Jurkat cells after 4 h, 5 h and 7 h (the first row of panels A and B), when compared with untreated cells. The results shown in FIG. 23, suggests that under normal circumstances, Bik is degraded rapidly by the proteasome. Blocking of this degradation by a proteasome inhibitor, allows the pro-apoptotic Bcl-2 member to accumulate. The accumulation of Bik may possibly tip the balance between pro- and anti-apoptotic factors favoring apoptosis.

The Effect of Overexpression of Bcl-xL, an Anti-Apoptotic Bcl-2 Family Member

The human B cell line Namalwa stably transfected with an anti-apoptotic Bcl-2 family member Bcl-xL, was shown to be more resistant to the proteasome inhibitor lactacystin than the untransfected, wild type Namalwa cells. The results shown in FIG. 24 indicate that the transfected cells have demonstrably less DNA fragmentation at the different intervals and lactacystin concentrations tested. This suggests that the overexpression of Bcl-xL protein has probably counteracted the effect of the accumulation of the pro-apoptotic Bik. In this manner the Namalwa cells are somewhat protected from undergoing apoptosis.

In an additional experiment, Jurkat cells, wild type Namalwa cells and Bcl-xL transfected Namalwa cells were treated with staurosporine and lactacystin for 6 H. Proteins from the mitochondrial fraction of these cells were analyzed by immunoblotting for the amount of Bik, Bcl-xL, Bax, and Bak. The results summarized in FIG. 25, show that Bik accumulates in the Namalwa cells (panel B, lane 3) and Jurkat cells (panel A lane 2) after a 6 hour lactacystin treatment. This accumulation is due to the inhibition of proteasome activity and indicates that the degradation of Bik via the proteasome is a general phenomenon. The elevated amount of Bik, is likely a mechanism of lactacystin-induced apoptosis in the Jurkat and Namalwa cells. The accumulation of Bik was only observed in the lactacystin-treated but not in staurosporine treated cells, eventhough staurosporine could equally induce apoptosis in these cells. The expression of exogenous anti-apoptotic Bcl-2 member Bcl-xL as expected, was not detected in Jurkat cells and wild type Namalwa cells (panels A and B). The Bcl-xL overexpression was obvious in the transfected Namalwa cells (panel C). Moreover, there was an accumulation of Bcl-xL after lactacystin treatment, showing that under normal circumstances the degradation of Bcl-xL, like Bic is also rapid and depends on proteasome activity. These results suggest that the Bcl-xL-transfected Namalwa cells have two mechanisms to protect them from proteasome inhibitor-induced apoptosis. First the overexpression of the anti-apoptotic Bcl-xL changes the balance between pro- and anti-apoptotic factors and favors the anti-apoptotic factors. Second, after treatment with lactacystin, there is an accumulation of Bcl-xL which imparts additional weight to the anti-apoptotic factors.

Thus, the balance between the pro- and anti-apoptotic factors in cells is crucial in deciding the fate of these cells. Certain apoptosis-related factors have a short half life and their degradation is via the proteasome machinery. Therefore, modulating the proteasome activity with proteasome inhibitors is a useful way to control the balance between the pro- and anti-apoptotic factors. This control provides the means to induce cells into apoptosis or continued survival.

Accordingly, it is an additional embodiment of this invention to provide the means to balance between pro-apoptotic and anti-apoptotic factors in a cell using proteasome inhibitors, particularly lactacystin.

DPBA is Effective in Treating Ongoing Heart Allograft Rejection in Mice

The proteasome inhibitor DPBA could effectively reverse the ongoing rejection. With a short-term treatment between day 3 and 6, the graft survival was prolonged to more than 13 days and is still counting.

The present invention is illustrated in further detail by the following non-limiting examples.

### EXAMPLE 1

Reagents

RPMI 1640, FCS, penicillin-streptomycin, and L-glutamine were purchased from Life Technologies (Burlington, Ontario, Canada). Lymphoprep was purchased from NYCOMED (Oslo, Norway). PHA, hydroxyurea, nocodazole, and histone H1 were from Sigma (St. Louis, Mo.). Staphylococcus aureus Cowan I (SAC) were obtained from Calbiochem (La Jolla, Calif.), and lactacystin from Dr. E. J. Corey (25). Human rIL-2 was from La Roche (Nutley, N.J.), and anti-CD3 mAb OKT3 was from ATCC (Rockville, Md.). FITC-conjugated anti-CD3 mAb(clone SFCIRW2-8C8) and PE-conjugated anti-CD25 mAb (clone IHT44H3) were from Coulter (Miami, Fla.). Anti-CD28 mAb (clone 9.3) was a gift from Dr. P. Linsley (26). A fluorogenic chymotrypsin substrate SLLVY-MCA was from Peninsula Laboratories (Belmont, Calif.). Rabbit antisera against cyclin A, Cyclin E, p27^{Kip1}, p21^{Cip1}, CDK2 and CDK4 were purchased from Santa Cruz Biotech (Santa Cruz, Calif.). [γ-³²p]ATP (3000 μCi/mmol) and [¹²⁵I] protein A (30 mCi/mg protein) were ordered from Amersham (Oakville, Ontario, Canada), and [Methyl-³H] thymidine (2 Ci/mmol) was from ICN (Irvine, Calif.).

Cell Culture

Peripheral blood mononuclear cells (PBMC) and tonsillar T cells were prepared as described before (Luo et al., 1992, Transplantation 53:1071; Luo et al., 1993, Clin. & Exp. Immunol. 94:371). The cells were cultured in RPMI 1640 supplemented with 10% FCS, L-glutamine and antibiotics. ³H-thymidine uptake was carried out as described previously (Luo et al., 1992, supra; Luo et al., 1993, supra).

DNA Fragmentation Assay

The assay was performed according to a protocol described by Liu et al (Liu et al., 1997, Cell. 89:175) with some modifications. Briefly, 2-6 million cells were re-suspended in 50 μl PBS followed by 300 μl lysis buffer (100 mM Tris-HCl, pH 8.0, 5 mM EDTA, 0.2 M NaCl. 0.2% w/v SDS, and 0.2 mg/ml proteinase K). After overnight incubation at 37° C., 350 μl of 3M NaCl was added to the mixture and cell debris was removed by centrifugation at 13000 g for 20 min at room temperature. DNA in the supernatant was precipitated with an equal volume of 100% ethanol. The pellet was washed with cold 70% ethanol and then dissolved in 20 μl of TE containing 0.2 mg/ml RNase A. After incubation at 37° C. for 2 h, the DNA was resolved on 2% agarose gel and visualized with ethidium bromide staining.

Electron Microscopy

T cells and Jurkat cells were examined by electron microscopy as described by Tsao and Duguid (Tsao et al., 1987, Exp. Cell Res. 168:365).

Flow Cytometry for IL-2Rα

Two-color staining with FITC-anti-CD3 and PE-anti-CD25 was performed on tonsillar T cells. The method was described before (Luo et al., 1993, supra).

Proteinase Assay

Jurkat cells were cultured with various treatments and were harvested and sonicated in 300 μl PBS on ice for 40 sec. Twenty micrograms of protein per sample from the cleared lysates were supplemented to 100 μl with 0.1M Tris buffer (pH 8.2). The fluorogenic chymotrypsin substrate sLLVY-MCA was added at a final concentration of 10 nM. The samples were incubated at 37° C. for 15 min and the reaction was terminated by adding 4 μl 2.5M HCl. The samples were then diluted to 2 ml with 0.1M Tris pH 8.2, and measured for their fluorescence intensity by a PTI fluorometer (Photo Technology International, South Brunswick, N.J.). The excitation wavelength was 380 nm, and the emission wavelength 440 nm.

Cell Cycle Synchronization of T Cells and Jurkat Cells

Tonsillar T cells were cultured in the presence of 2 μg/ml PHA and 1 mM hydroxyurea for 40 h. The cells thus treated were synchronized at the G₁/S phase. The synchronization was released by washing out hydroxyurea, and the cells were cultured in medium for additional 6-22 h according to the need of each experiment. The synchronization of Jurkat cells was described in our previous publication (Shan et al., 1994, Int. Immunol. 6:739). Briefly, the Jurkat cells were starved in isoleucine deficient medium for 24 h followed by 16 h treatment with 2 mM hydroxyurea (HU). Cells thus treated were synchronized at the G₁/S boundary. For synchronization at the G₂/M boundary, the G₁/S synchronized cells were released from hydroxyurea and cultured in regular medium for 6 h, and then treated with 0.1 μg/ml nocodazole for 16 h. The cells were then synchronized at the G₂/M boundary.

Cell Cycle Analysis

Flow cytometry was employed for cell cycle analysis for T cells and Jurkat cells as described before (Shan et al., 1994, supra) using propidium iodide staining.

Immunoblotting

Immunoblotting was employed to evaluate the levels of Cyclin E, cyclin A, p21^{Cip1} and p27^{Kip1}. The general protocol was described in our previous publication (Chen et al., 1996, supra). Briefly, lymphocytes were lysed in the presence of proteinase inhibitors. The cleared lysates were quantitated for protein concentrations. An equal amount of lysate proteins (40 μg) of each sample was resolved by 10% SDS-PAGE and was transferred to PVDF membranes (Millipore, Bedford, Mass.). The membranes were then blocked with 5% milk, and hybridized with rabbit antisera against Cyclin E, cyclin A, p27^{Kip1} and p21^{Cip1} at dilutions suggested by the manufacturer. The signals on the membrane were detected by [¹²⁵I]-protein A followed by autoradiography.

Immunoprecipitation and the Kinase Assay

Lymphocytes were lysed by a lysis buffer as used in the immunoblotting (Chen et al., 1996, supra), and cleared lysates were quantitated for their protein content. For immunoprecipitation, 50 μl of rabbit antisera against CDK2, CDK4 or Cyclin E were added to the lysates equivalent to 20 or 40 μg protein depending on the experiment. After 2 h incubation at 4° C., the immune complexes were recovered by protein A-conjugated Sepharose (Pharmacia Biotech, Montreal, Quebec, Canada). The immune complexes bound to protein A-Sepharose were extensively washed in a lysis buffer without detergents or EDTA, and resuspended in 50 μl of kinase reaction buffer (100 mM NaCl, 20 mM HEPES, pH7.S, 5 mM MnCl₂, 5 mM MgCl₂, 25 μM cold ATP, 2.5 μCi [γ−³²p] ATP, and 3 μg histone H1 as a substrate). The reaction was carried out for 10 min at room temperature, and stopped by adding the SDS-PAGE loading buffer. After boiling for 3 min, the samples were subjected to 10% SDS-PAGE. The proteins were then transferred to PVDF membranes and the signals were detected by autoradiography.

### EXAMPLE 2

### Assays Measuring Nitric Oxide Production

Macrophage Preparation and Culture

BALB/c mice were injected i.p. with 3 ml of 3% thioglycollate broth. Three days later, peritoneal exudate macrophages of the mice were harvested and washed at 170 g for 10 min at 4° C. The macrophages were cultured in Teflon vials (2 cm in diameter) at 4×10⁶/2 ml with various reagents (LPS, 2 μg/ml; IFNγ, 100 u/ml; LAC, 0.62-5 μM for the nitric oxide assay and 5 μM for the Northern blot assay).

Nitric Oxide Measurement

The nitrite concentration in the culture supernatant was measured as a way to indirectly reflect the nitric oxide level following a method described by Ding et al (Ding et al., 1988, J. Immunology 141:2407). Release of reactive nitrogen intermediates and reactive oxygen intermediates form mouse peritoneal macrophages: comparison of activation cytokines and evidence for independent production. Briefly, 100 μl of supernatants collected from 48 h macrophage cultures was incubated with an equal volume of the Griess reagent (1% sulfanimide/0.1% naphthylethylene diamine dihydrochloride/2.5% H₃PO₄) at room temperature for 10 min in 96-well microtitration plates, the O.D. was measured at 550 nm. Sodium nitrite of various concentrations were used to construct standard curves.

Northern Blot Analysis of iNOS Expression

The expression of inducible nitric oxide synthase at the mRNA level was analyzed by Northern blot as described in our previous publication (Shan et al., 1994, supra). After an overnight culture, the mouse macrophages were harvested and their total cellular RNA was extracted with the guanidine/CsCl method. The RNA (10 μg/lane) was resolved in 1% agarose-formaldehyde gels and blotted onto nylon membranes. A 562-bp fragment corresponding to the mouse iNOS cDNA (Xie et al., 1992, Science 256:225) was obtained by reverse transcription/PCR using the mouse macrophages total RNA as templates. The fragment was labeled with ³²p with random primers and used as a probe for the Northern blot.

### EXAMPLE 3

### Respiration of Jurkat Cells

Preparation of Mitochondria

Rat liver of rat kidney proximal tubules mitochondria were isolated by differential centrifugation in a medium containing 250 mM sucrose, 1 mM HEPES-Tris, 250 μM EDTA (pH 7.5). The last washing of the mitochondria was performed in the same medium without EDTA. Protein concentration of the mitochondrial suspension was measured after solubilization of the membranes in 0.1% SDS with the Pierce-BCA (bicinchroninic acid) protein assay reagent (Pierce, Rockford, Ill., U.S.A.), using bovine serum albumin as a standard.

Respiration Measurements

The Jurkat Cells (JC) (30×10⁶/ml) or rat kidney proximal tubules mitochondria (RKM) (0.5 mg of protein/ml) were incubated in 1 ml measuring chamber at 37° C. in a respiration buffer containing 200 mM sucrose, 5 mM MgCl₂, 5 mM KH₂PO₄, and 30 mM HEPES-Tris (pH 7.5). During respiration experiments following substrates and inhibitors were used: 0.005% Digitonine (Dig); 10 mM Succinate (Suc); 1 mM Ascorbate (Asc); 0.4 mM tetramethyl-p- phenylenediamine (TMPD); 1 μM CCCP, 1 μM FCCP; 0.1 μM Rotenone (Rot); 50 nM Antimycin A (Anti); 1 mM KCN; 100 μM Cytochrome C (Cyt C). The respiration rate of the Jurkat Cells and mitochondria was measured polarographically with a Clarke oxygen electrode (Yellow Springs Instruments, Yellow Springs, Ohio, U.S.A.) using 1 ml thermojacketed chamber. Oxygen concentration was calibrated with air-saturated buffer using Hypoxanthine—Xanthine Oxidase—Catalase system (“chemical zero”). Oxygen consumption was continuously recorded using a “MacLab/8” (Analog Digital Instruments, U.S.A.) connected to a Macintosh SE computer and the MacLab Chart v.3.3.4 software. Rates of oxygen consumption are expressed as ng-atoms of oxygen/min.

### EXAMPLE 4

### The Effect of Immunosuppressive Drugs

Cell Culture

PBMC were prepared by Lymphoprep gradient as described before (Luo et al., 1993, supra; Shan et al., 1994, supra). Tonsillar T cells were prepared by one cycle of SRBC rosetting and such preparation contained 80-85% CD3⁺ cells. The remaining tonsillar cells were referred to as the tonsillar B cells, which were about 90% CD20⁺ cells.

Northern Blot Analysis

The method is described in our previous publication (Shan et al., 1994, supra). Tissue or lymphocyte total RNA was extracted with the guanidine/CsCl method and used in the Northern blot analysis. A 358-bp fragment corresponding to positions −14 to 314 of the PA28β cDNA (Ahn et al., 1995, FEBS Lett. 366:37) from clone 5F2 was labeled with ³²p using random primers and was used as a probe for PA28β messages. A 400-bp fragment corresponding to positions between 267 and 666 of the PA28α cDNA (Realini et al., 1994, supra) was obtained with RT-PCR and was used as a probe for PA28α messages. The 5′ and 3′ primers for the RT-PCR were GAAGAAGGGGGAGGATGA and AGCATTGCGGATCTCCAT, respectively.

Immunoblotting

T cell lysates (40 μg protein/sample) were separated on 12% SDS-PAGE, and blotted onto PVDF membranes. The membranes were then hybridized with rabbit anti-PA28β antiserum (Ahn et al. 1996, J. Biol. Chem. 271:18237) followed by ¹²⁵I-protein A. Detailed methods were described previously (Chen et al., 1996, supra).

Confocal Immunoflurescent Microscopy

Cultured tonsillar T cells were stained with rabbit anti-PA28 antiserum (1:1000 dilution) or anti-PA28α antiserum (1:200 dilution) followed by biotin-conjugated goat anti-rabbit IgG (1:100 dilution, Boehringer Mannheim, Montreal, QC) and streptavidin-fluorescein. The immunofluorescence of whole cells was examined and quantified with confocal microscopy as detailed before (Chen et al., 1997, J. Immunol. 159:905).

Proteinase Assay

PBMC were cultured with or without PHA (2 μg/ml) and RAPA (10 nM). After 16 h-70 h, the cells were harvested and sonicated in 300 μl PBS on ice for 40 sec. Twenty micrograms of protein per sample from the cleared lysates were supplemented to 100 μl with 0.1 M Tris buffer (pH 8.2). A proteasome-specific inhibitor lactacystin (Omura et al., 1991, supra; Fentenay et al., 1995, supra) was added at a final concentration of 10 nM in some samples as indicated. The samples were incubated on ice for 15 min, and fluorogenic chymotrypsin substrate sLLVY-MCA was then added at a final concentration of 10 nM. The 20S proteasome, which was prepared as previously described (Friguet et al, 1994, J. Biol. Chem. 269:21639), was used as a positive control in place of cell lysates. The samples were incubated at 37° C. for 15 min and the reaction was terminated by adding 4 μl 2.5 M HCl. The samples were then diluted to 2 ml with 0.1 M Tris pH8.2, and measured for their fluorescent intensity by a PTI fluorometer (Photo Technology International, South Brunswick, N.J.). The excitation wavelength was 380 nm, and the emission wavelength 440 mn.

### EXAMPLE 5

### The Use of DPBA to Treat Allograft Rejection in Transplantation

Synthesis of DPBA

The applicant first synthesized DPBA (FIG. 26), and it had the expected inhibitory effect to the chymotrypsin-like activity of the 20S proteasome as shown in FIG. 27. The IC₅₀ for the inhibition of the chymotrypsin-like enzyme was about 20 nM. DPBA also potently inhibited proliferation of anti-CD3-stimulated T cells with IC₅₀ of about 18 nM (FIG. 28), which is consistent with DPBA's IC₅₀ in enzyme inhibition. This showed that DPBA, like LAC, is effective in inhibiting T cell activation and proliferation in vitro.

Use of DPBA in Mouse Model of Heart Transplantation

The applicant then used DPBA in treating allograft rejection in a mouse model. BALB/c (H-2^{d}) mice were used as donors and C57BL/6 (H-2^{b}) was used as recipients. Heterotropic heart transplantation was performed as described in our previous publication (Chen et al., 1996, supra). As shown in FIG. 29, the control group had mean survival rate (MST) of 7.3+0.5 (SD) days. When the recipients were administrated with straight 0.65 mg/kg/day, i.p. of DPBA for 16 days, the MST was more than 26.2+13 days. To mimic the clinical regimen of immunosuppressants, the applicant also tried a short-term high dosage of DPBA immediately after the transplantation (1 mg/kg/day, i.p. for 4 days from day 1 to day 4 post transplantation), followed by a low dosage (0.5 mg/kg/day, i.p. from day 5 to day 16). With this regimen, the MST is more than 22.8+9.8 days and has a tendency of being better than the first group. The mice appear healthy during or after the drug administration. These results for the first time show that a proteasome inhibitor can be used as an effective immunosuppressant in organ transplantation, and the applicants have proved that there exists a therapeutic dose window between the effective and toxic dosages of the proteasome inhibitor.

Treatment of Ongoing Rejection in the Mouse Heart Transplantation Model

Proteasome inhibitors when added at the late G1 phase can suppress proliferation and even induce apoptosis of the activated T cells. This suggests that the inhibitors could treat ongoing rejection. This possibility was tested in mouse heart transplantation model. The recipients were given no immunosuppressants for 72 h after the transplantation to allow the rejection response to proceed. Starting on day 3, i.e. 72 h after the operation, the mice were given DPBA at 1 mg/kg/day, i.p. for 4 days. As shown in FIG. 30, the MST is more than 13.2+1.78. The result suggests that the proteasome inhibitor will be useful in treating clinical rejection episodes, which are normally diagnosed when the rejection is ongoing. This new drug will be especially useful in patients who are resistant to commonly used immunosuppressants such as CyA, azathioprine, and glucocorticoids.

The applicant has for the first time successfully used a proteasome inhibitor to prevent allograft rejection. The proteasomes were thought to be humble “garbage collectors” to degrade cellular proteins in an unregulated way. The applicant has raised a novel concept and proved that the proteasome plays critical roles in immune regulation and the proteasome inhibitors can be used as novel immunosuppressants in organ transplantation. The applicant have proved that there is a therapeutic dose window for the proteasome inhibitors in vivo, and the inhibitors are effective in treating ongoing graft rejection. Thus, the proteasome inhibitors, as represented by DPBA, are a new class of immunosuppressants. The usefulness of these class of immunosuppressants are in following three aspects: 1) They can be used alone, or in combination with other immunosuppressive drugs in allo or xeno organ transplantation; 2) They are especially useful in controlling clinical rejection episodes, which are normally diagnosed when the T cells are already activated, and are less responsive or resistant to conventional immunosuppressants; 3) They could be used in inducing long-term graft survival by clonal deletion of alloantigen- or xenoantigen-specific T cells when administered after the activation of these cells; 4) By replacing the amino acid residues in the DPBA, one could generate proteasome inhibitors competitively inhibiting other protease activities of the proteasome, an some of them might have better therapeutic effects than the model DPBA used in this study. For example, one could replace the Phe and Leu in DPBA with other bulky hydrophobic amino acids to alter DPBA's inhibitory profile of the chymotrypsin-like activity of the proteasome; Lys and Arg can be used in the structure to generate inhibitors for the trypsin-like activity of the proteasome; Glu, branched amino acids, and small neutral amino acids could be used in the structure to generate inhibitors for the peptidylglutamyl peptide-hydrolyzing, branched chain amino-preferring, and small neutral amino acid-preferring activities, respectively.

The Use of DPBA in Organ Transplantation—Islet Graft in Streptozocin—Induced Diabetes in Mice.

The islets from syngeneic mice (isograft control) restored normal glycemia in diabetic mice, and the effect lasted more than 60 days as expected. The allogeneic islets were rejected in about 10 days in untreated mice, and the mice became diabetic after an initial dip of their blood sugar level (allograft control). When the allogeneic islets were transplanted to diabetic recipients along with DPBA treatment, the graft functionned normally beyond 60 days, indicating that the graft rejection was inhibited. This result demonstrates that proteasome inhibitors as exemplified by DPBA can be used in human islet transplantation to prevent graft rejection. FIG. 31 shows that a proteasome inhibitor such as DPBA inhibits the glucose elevation consequent to islet rejection.

Conclusion

The proteasome inhibitors, represented hereinabove by LAC and DPBA have shown a unique capacity to reverse an ongoing activity of blood cells. This reversal heretofore makes possible the treatment which selectively targets activated blood cells.

Although the present invention has been described hereinabove by way of preferred embodiments thereof, it can be modified, without departing from the spirit and nature of the subject invention. Any such modification is under the scope of this invention as defined in the appended claims.

What is claimed is:

1. A method for reversing an ongoing proliferation or activity, or both, of activated blood cells, which comprises the step of administering an effective amount of a proteasome inhibitor to an individual in need for such a treatment.

2. A method as defined in claim 1, wherein said individual suffers from an adverse immune response, inflammation, or septic shock.

3. A method as defined in claim 2, wherein said adverse immune response is an autoimmune disease or a graft rejection.

4. A method as defined in claim 1, further comprising the step of co-administering an immunosuppressive drug with said proteasome inhibitor.

5. A method as defined in claim 4, wherein said immunosuppressive drug is selected from the group consisting of cyclosporin A, rapamycin and FK506.

6. A method as defined in claim 1, which results into activated blood cells apoptosis.

7. A method as defined in claim 1, which results into inhibition of energy and oxygen supply to said activated blood cells.

8. A method as defined in claim 7, wherein said inhibition of energy and oxygen supply is caused by disrupting mitochondrial function in activated blood cells.

9. A method as defined in claim 7, wherein said inhibition of energy and oxygen supply is caused by disruption of nitric acid synthesis.

10. A method as defined in claim 1, wherein said proteasome inhibitor is lactacystin or dipeptide boronic acid (DPBA), or analogs thereof.

11. A method as defined in claim 10, wherein said proteasome inhibitor is lactacystin.

12. A method as defined in claim 10, wherein said proteasome inhibitor is DPBA.
